(19) United States
(12) Patent Application Publication
(10) Pub. No.: US 2012/0087986 A1
(43) Pub. Date: Apr. 12, 2012

(54) PHARMACEUTICAL FORMULATIONS COMPRISING DESVENLAFAXINE

(76) Inventors: Nagaraju Nagesh, Hyderabad (IN); Shrikant Vitthalrao Dhoke, Nagpur (IN); Sanjay Shekhranand Tripathi, Nagpur (IN); Radheshyam Kale, Nagpur (IN); Harshal Prabhakar Bhagwatwar, Hyderabad (IN); Venkateswarlu Vobalaboina, Hyderabad (IN); Venkata Nookaraju Sreedharala, Hyderabad (IN); Debashis Dash, Hyderabad (IN)

(21) Appl. No.: 13/269,752

(22) Filed: Oct. 10, 2011

Related U.S. Application Data
(60) Provisional application No. 61/416,983, filed on Nov. 24, 2010.

(30) Foreign Application Priority Data
Oct. 11, 2010 (IN) 3008/CHE/2010

(51) Int. Cl. A61K 9/14; A61P 25/24; A61K 9/00; A61K 31/137

(52) U.S. Cl. 424/497; 514/653; 424/400

(57) ABSTRACT

Pharmaceutical formulations comprising desvenlafaxine, processes for preparing formulations comprising desvenlafaxine, and therapeutic uses and methods of treatment employing formulations comprising desvenlafaxine.

### INTRODUCTION

Aspects of the present application relate to formulations comprising desvenlafaxine, and processes for preparing the formulations. In particular aspects, the present application relates to stabilized formulations comprising desvenlafaxine succinate, and processes for preparing the same. Aspects of the application further relate to therapeutic uses and methods of treatment employing formulations comprising desvenlafaxine.

“O-desmethylvenlafaxine,” “desvenlafaxine,” and “ODV” are names used for the drug compound having chemical names: 1-[2-dimethylamine (4-hydroxyphenyl)ethyl]cyclohexanol; or RS-4-[2-dimethylamino-1-(1-hydroxycyclohexyl)ethyl]phenol; and represented by structural Formula A.

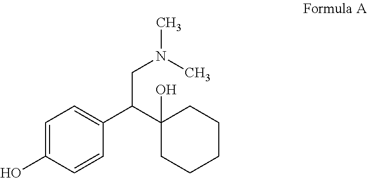

Desmethylvenlafaxine is prescribed for treating major depressive disorders. Desmethylvenlafaxine, the major metabolite of venlafaxine, selectively blocks the reuptake of serotonin and norepinephrine. The currently marketed PRISTIQ® sustained-release tablets contain 50 mg or 100 mg of desvenlafaxine base, in the form of desvenlafaxine succinate monohydrate, for oral administration. PRISTIQ tablets contain the inactive ingredients hypromellose, microcrystalline cellulose, talc, magnesium stearate, with coatings containing polyvinyl alcohol, polyethylene glycol, talc, titanium dioxide, iron oxide, and (only for the 100 mg strength) FD&C yellow #6.

A major depressive episode (DSM-IV) implies a prominent and relatively persistent (nearly every day for at least 2 weeks) depressed or dysphoric mood that usually interferes with daily functioning, and includes at least five of the following symptoms: depressed mood, loss of interest in usual activities, significant change in weight and/or appetite, insomnia or hypersomnia, psychomotor agitation or retardation, increased fatigue, feelings of guilt or worthlessness, slowed thinking or impaired concentration, or a suicide attempt or suicidal ideation.

U.S. Pat. No. 4,535,186 discloses desmethylvenlafaxine and its pharmaceutically acceptable salts. U.S. Pat. No. 6,673,838 discloses the succinate salt of desvenlafaxine, and also describes four polymorphic forms of ODV succinate (Forms I, II, III, and IV) and an amorphous form, as well as a composition, method of use and pharmacokinetic parameters. U.S. Pat. No. 7,291,347 discloses tablet and capsule dosage forms of desvenlafaxine.

U.S. Patent Application Publication No. 2009/0018208 discloses a pharmaceutical composition comprising O-desmethylvenlafaxine succinate, wherein O-desmethylvenlafaxine is present in amounts about 75 mg and 30-90 mg.

International Application Publication No. WO 2009/075677 discloses a method of treating a patient suffering from major depressive disorder, comprising administering to a patient in need thereof a daily dose of about 50 mg of O-desmethylvenlafaxine or an equivalent amount of a pharmaceutically acceptable salt thereof.

U.S. Patent Application Publication No. 2005/0197392 discloses a pharmaceutical composition comprising (±)-O-desmethylvenlafaxine succinate and a pharmaceutically acceptable carrier or excipient, wherein (±)-O-desmethylvenlafaxine is present in amounts about 50 mg and 100 mg. Its equivalent U.S. Patent Application Publication Nos. 2004/0106576, 2008/0269166, and 2008/0132578 also disclose the use of such compositions for the treatment of several disorders.

U.S. Patent Application Publication No. 2006/0193912 discloses a controlled release oral dosage form comprising: a therapeutically effective amount of O-desmethylvenlafaxine or a pharmaceutically acceptable salt thereof and a release controlling material; wherein the amount of O-desmethylvenlafaxine or pharmaceutically acceptable salt thereof released at 1 hour in 900 mL of 0.1N HCl (pH 1.5) with 40% ethanol, using USP apparatus 2 at 50 rpm, is within 25% of the amount of O-desmethylvenlafaxine or pharmaceutically acceptable salt thereof released at 1 hour in 900 mL of 0.1 N HCl (pH 1.5) using USP apparatus 2 at 50 rpm.

International Application Publication No. WO 2009/009665 discloses an amorphous solid dispersion of desvenlafaxine succinate and PVP. However, the application does not disclose pharmaceutical compositions of desvenlafaxine succinate that provide modified drug release.

Several patents and other publications disclose different crystalline forms of desvenlafaxine, salts, and pharmaceutical compositions containing the same. Several other publications disclose methods of use of desvenlafaxine for various indications.

There remains a need for pharmaceutical formulations comprising desvenlafaxine or pharmaceutically acceptable salts, esters, hydrates, solvates, derivatives or single enantiomer thereof for providing effective plasma concentrations of the active agent, that are easy to manufacture.

### SUMMARY

Aspects of the present application relate to formulations comprising desvenlafaxine, including any pharmaceutically acceptable salts, esters, hydrates, solvates, and derivatives thereof, and processes for preparing the same.

In embodiments, the present application relates to pharmaceutical formulations comprising desvenlafaxine succinate, together with one or more excipients.

In embodiments, the present application relates to pharmaceutical formulations comprising amorphous desvenlafaxine succinate, together with one or more excipients.

In embodiments, the present application provides pharmaceutical compositions comprising solid dispersions or premixes of desvenlafaxine succinate.

In embodiments, the present application provides pharmaceutical formulations comprising solid dispersions or premixes of desvenlafaxine succinate, together with one or more pharmaceutically acceptable excipients.

In embodiments, the present application provides pharmaceutical formulations comprising solid dispersions or premixes of desvenlafaxine succinate, together with at least one release controlling substance and one or more other pharmaceutically acceptable excipients.

In embodiments, the present application relates to pharmaceutical formulations comprising solid dispersions of desvenlafaxine succinate, wherein the desvenlafaxine succinate is in a substantially amorphous form, and wherein the substantially amorphous form is retained during manufacturing and during storage of formulations for commercially relevant times.

In embodiments, the present application relates to stable pharmaceutical formulations comprising amorphous desmethylvenlafaxine succinate, present in the form of a premix or solid dispersion, one or more release rate controlling polymers, and optionally one or more pharmaceutically acceptable excipients, wherein the amorphous nature of desmethylvenlafaxine succinate is retained during preparation of the formulations and also throughout the intended shelf-life of the formulations.

In embodiments, the present application relates to pharmaceutical formulations comprising solid dispersions of desvenlafaxine succinate, prepared with a hydrophilic excipient.

In embodiments, the present application relates to pharmaceutical formulations comprising solid dispersions of desvenlafaxine succinate, prepared with a hydrophobic excipient.

In embodiments, the present application relates to pharmaceutical formulations comprising solid dispersions of desvenlafaxine succinate and a polyvinylpyrrolidone in weight ratios of 1:10 to 10:1.

An aspect of the present application provides polymorphically stable forms of desvenlafaxine. In embodiments, polymorphically stable forms of desvenlafaxine comprise amorphous desvenlafaxine together with at least one pharmaceutically acceptable excipient.

In embodiments, the present application relates to modified release pharmaceutical formulations comprising desvenlafaxine succinate, together with at least one release controlling excipient, optionally with one or more other pharmaceutically acceptable excipients.

Desvenlafaxine modified release formulations in embodiments of the present application are intended to provide effective plasma concentrations of the active agent for extended durations of time, following administration to a subject in need thereof.

In embodiments, the present application provides processes for preparing formulations comprising desvenlafaxine or pharmaceutically acceptable salts, esters, hydrates, solvates, derivatives, or single enantiomers thereof.

In embodiments, the present application provides process for preparing stable pharmaceutical formulations comprising amorphous desmethylvenlafaxine succinate, present in the form of a premix or solid dispersion, one or more release rate controlling polymers, and optionally one or more other pharmaceutically acceptable excipients, wherein the amorphous nature of desmethylvenlafaxine succinate is retained during preparation of the formulations and also throughout the intended shelf-life of the formulations.

In aspects, the present application provides therapeutic uses and methods of treatment employing formulations comprising desvenlafaxine. In embodiments, the present application provides methods of prophylaxis, amelioration, or treating depression, such as major depressive disorder in a subject in need thereof, by administering a therapeutically effective amount of a pharmaceutical formulation comprising amorphous desmethylvenlafaxine succinate, present in the form of a premix or solid dispersion, one or more release rate controlling polymers, and optionally one or more other pharmaceutically acceptable excipients, wherein the amorphous nature of desmethylvenlafaxine succinate is retained during preparation of the formulations and also throughout the intended shelf-life of the formulations.

### BRIEF DESCRIPTION OF THE DRAWINGS

FIG. 1 illustrates powder X-ray diffraction (PXRD) patterns of materials from Example 2.

FIG. 2 illustrates PXRD patterns of materials from Example 14A.

FIG. 3 illustrates PXRD patterns of materials from Example 14B.

FIG. 4 illustrates PXRD patterns of materials from Example 14C.

FIG. 5 illustrates PXRD patterns of materials from Example 15A.

FIG. 6 illustrates PXRD patterns of materials from Example 15B.

In the figures, the y-axis shows intensity units and the x-axis is the 2θ angle, in degrees.

### DETAILED DESCRIPTION

As used herein, the term “desvenlafaxine” includes the compound desvenlafaxine base, pharmaceutically acceptable salts, esters, and prodrugs thereof, the active metabolites of desvenlafaxine and the prodrugs thereof, single enantiomers thereof, and any of their polymorphs, solvates, and hydrates.

The terms “pharmaceutically acceptable salt or ester” as used herein refers to salts or esters that are known to be non-toxic and are commonly used in pharmaceutical practice.

In the present application, desvenlafaxine can be used in any crystalline form, amorphous form, or combinations thereof.

The term “excipient” or “pharmaceutically acceptable excipient” means a component of a pharmaceutical product that is not an active ingredient, such as a filler, diluent, carrier, etc. The excipients that are useful in preparing a pharmaceutical composition are generally safe, non-toxic and neither biologically nor otherwise undesirable, and are acceptable for veterinary use as well as human pharmaceutical use. An “excipient” includes both one and more than one substance.

The term “stability” as used in the description includes both physical and chemical stability. The term “physical stability” refers to maintaining the polymorphic form of desvenlafaxine, such as crystalline, amorphous, or mixtures thereof, and “chemical stability” refers to maintaining acceptable concentrations of drug compound-related impurities.

The term “rate controlling” as used herein refers to any excipient substance that can alter or modify the drug release time, rate, and extent in any manner from a composition or formulation, such as, for example controlling, sustaining, modifying, prolonging, or delaying the drug release.

An aspect of the present application provides compositions containing substantially pure desvenlafaxine, which compositions are simple, cost-effective, do not involve toxic and hazardous solvents, and also are easy to make on a commercial scale.

An aspect of the present application provides compositions comprising stable amorphous solid dispersions of desvenlafaxine succinate and processes for their preparation.

An aspect of the present application provides compositions prepared using crystalline forms of desvenlafaxine succinate such as Form V and Form VI, as described in International Application Publication No. WO 2009/009665, and processes for their preparation.

In embodiments, desvenlafaxine used to make compositions or contained in compositions is in the forms of amorphous, crystalline, or mixtures thereof. In embodiments, desvenlafaxine used as the active agent is in a substantially amorphous form, which form is substantially retained during the manufacturing of the composition and also during storage. In embodiments, desvenlafaxine used as the starting active agent is in a substantially crystalline form, which form is substantially retained during the manufacturing of the composition and also during storage.

Polymorphic forms of desvenlafaxine, including crystalline and amorphous forms, can be identified using analytical methods such as PXRD, infrared spectrophotometry, thermal analysis techniques, etc., all of which are familiar to those skilled in the art. All PXRD results described herein are obtained using copper Kα radiation.

In embodiments, the present application provides pharmaceutical formulations comprising desvenlafaxine succinate, together with one or more excipients. In embodiments, the present application provides pharmaceutical formulations comprising amorphous desvenlafaxine succinate, together with one or more excipients.

In embodiments, the present application provides pharmaceutical compositions in the form of solid dispersions or premixes of desvenlafaxine succinate.

In embodiments, the present application provides pharmaceutical formulations comprising solid dispersions or premixes of desvenlafaxine succinate, together with one or more pharmaceutically acceptable excipients.

In embodiments, the present application provides pharmaceutical formulations comprising solid dispersions or premixes of desvenlafaxine succinate, together with at least one release controlling substance and one or more other pharmaceutically acceptable excipients.

In embodiments, the present application provides pharmaceutical formulations comprising solid dispersions of desvenlafaxine succinate, wherein the desvenlafaxine succinate is in a substantially amorphous form, and wherein the substantially amorphous form is retained during manufacturing and storage.

In embodiments, the present application provides pharmaceutical formulations comprising solid dispersions of desvenlafaxine succinate, prepared with a hydrophilic compound.

In embodiments, a hydrophilic substance useful in making a solid dispersion of premix includes, but is not limited to, a polyvinylpyrrolidone, hydroxypropyl methylcellulose, polyethylene glycol, and the like, including any mixtures thereof.

In embodiments, the present application provides pharmaceutical formulations comprising solid dispersions or premixes of desvenlafaxine succinate, prepared with a polyvinylpyrrolidone (“povidone” or “PVP”) in weight ratios of drug to polymer about 1:10 to 10:1.

In embodiments, the present application provides pharmaceutical formulations comprising solid dispersions or premix compositions of desvenlafaxine succinate, prepared with a polyvinylpyrrolidone in a weight ratio of drug to polymer about 1:1.

An aspect of the present application provides polymorphically stable forms of desvenlafaxine.

In embodiments, the present application relates to modified release pharmaceutical formulations comprising desvenlafaxine succinate, together with at least one release controlling substance, and optionally also including one or more other pharmaceutically acceptable excipients.

In embodiments, stable desvenlafaxine formulations of the present application are in the form of tablets.

In embodiments, stable desvenlafaxine formulations of the present application are in the form of multi-particulates. In embodiments, ‘multi-particulates’ according to the present application may be in the form of powders, granules, pellets, spheroids, extrudates, mini-tablets, and the like.

In embodiments, stable desvenlafaxine formulations of the present application are in the form of multi-particulates, made into unit dosage forms such as capsules.

In embodiments, desvenlafaxine formulations of the present application are in the form of pellets or mini-tablets, filled into capsules.

The different physicochemical properties of the active ingredient as well as of excipients are to be considered, as these properties affect processing and formulation properties. Various important physicochemical properties include, but are not limited to, particle sizes, density (bulk density and tapped density), compressibility index, Hausner's ratio, angle of repose, etc. Particle sizes of active pharmaceutical ingredient can affect the solid dosage form in numerous ways. For example, content uniformity (CU) of pharmaceutical dosage units can be affected by particle sizes and size distributions. This will be even more critical for low-dose drugs and satisfactory dosage units of low doses cannot be manufactured from a drug that does not meet certain particle size and size distribution specifications. Also particle sizes play an important role in dissolution of active ingredient from the final dosage forms for certain drugs like desvenlafaxine because of their poor solubility. Hence, these physicochemical properties not only affect the processes of preparing the pharmaceutical compositions but also affect the performance of the pharmaceutical product, both in vitro and in vivo.

The selection of appropriate particles sizes of desvenlafaxine as well as of excipients is within the scope of the application. The D₁₀, D₅₀, and D₉₀ values are useful ways for indicating a particle size distribution. D₉₀ is the size value where at least 90 volume percent of the particles have sizes smaller than the value. Likewise, a D₁₀ value refers to 10 volume percent of the particles having sizes smaller than the value. A D₅₀ value refers to 50 volume percent of the particles having sizes smaller than the value, and a D[4,3] value refers to the mean particle size. Methods for determining D₁₀, D₅₀, D₉₀, and D[4,3] include laser diffraction techniques, such as using equipment sold by Malvern Instruments Ltd., Malvern, Worcestershire, United Kingdom, or by Horiba.

In embodiments, compositions of the present application are prepared using desvenlafaxine or a pharmaceutically acceptable form of desvenlafaxine having particle size distributions where: D₉₀ is about 1 μm to about 1000 μm, or about 1 μm to about 500 μm, or about 10 μm to about 250 μm; and D₅₀ is from about 1 μm to about 500 μm, or about 1 μm to about 250 μm, or about 1 μm to about 100 μm.

In embodiments, compositions of the present application is in the form of particles which are further made into suitable dosage forms, wherein the particles have Carr's index values in the range of about 1-40%. This indicates superior handling capabilities during processing into pharmaceutical dosage forms. Flowability of materials can be measured and represented using the Carr's Index. The Carr's Index is the percent ratio of the difference between tapped density and bulk density to tapped density, mathematically described as:

Carr's Index=[(Tapped density−Bulk density)÷Tapped density]×100.

The densities can be determined using test method 616 “Bulk Density and Tapped Density” of United States Pharmacopeia 29, United States Pharmacopeial Convention, Inc., Rockville, Md., U.S.A., 2005. Carr's Index values below about 15% represent materials with very good flow properties and values above about 40% represent materials with very poor flow properties.

In embodiments, desvenlafaxine-containing particles of the present application have particle sizes in the range of about 500 μm to about 2000 μm.

In embodiments, the present application provides stabilized modified release formulations comprising desvenlafaxine or pharmaceutically acceptable salts thereof, wherein the formulations release about 70-90% of contained active ingredient, within about 12 hours after immersion into 900 mL of a 0.9% NaCl dissolution medium, using test method 711 “Dissolution” in United States Pharmacopeia 29, United States Pharmacopeial Convention, Inc., 2005 (“USP”), and type 2 apparatus.

Among the various degradants and drug-related impurities that may be present in a desvenlafaxine-containing formulation, the compounds below, having Formula I to Formula VII, have been identified.

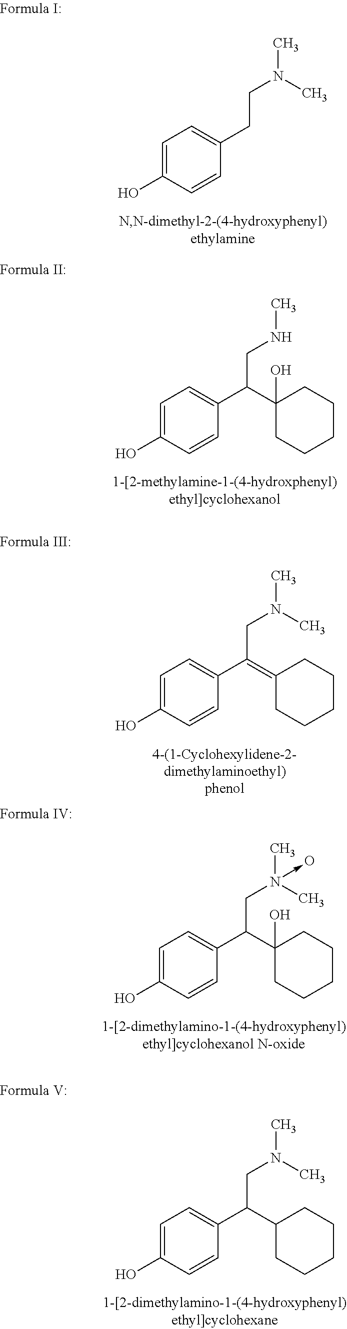

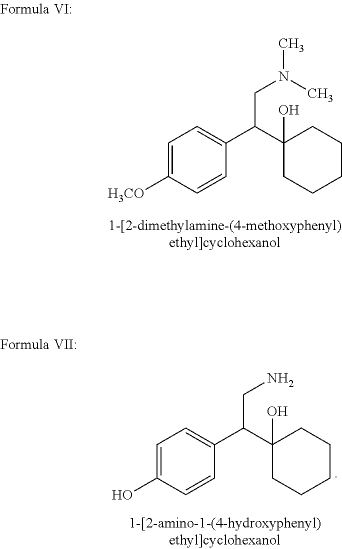

Desvenlafaxine and its impurities can be analyzed using high performance liquid chromatography (HPLC) with gradient elution. In a particular analysis, two mobile phases are used: mobile phase A is a phosphate buffer (pH 3.0) and acetonitrile, in the volume ratio of 90:10, respectively; and mobile phase B is a phosphate buffer (pH 3.0), acetonitrile, and methanol, in the volume ratio of 30:50:20, respectively. The various chromatographic parameters are as follows:

Column: C18, 250×4.6 mm, 5 μm.

Flow rate: 0.5-2 mL.

UV detector wavelength: 225 nm.

Column temperature: 20-40° C.

Injection volume: 5-25 μL.

Run time: 75 minutes.

Elution gradient as shown in Table 1.

TABLE 1

| | Mobile Phase A | Mobile Phase B |
|---|---|---|
| Minutes | (%) | (%) |
| 0 | 100 | 0 |
| 7 | 100 | 0 |
| 12 | 80 | 20 |
| 25 | 70 | 30 |
| 40 | 40 | 60 |
| 65 | 20 | 80 |
| 67 | 100 | 0 |
| 75 | 100 | 0 |

An example of relative retention times observed for impurities of Formulas I-VII and desvenlafaxine is 0.346, 0.955, 1.493, 1.132, 1.679, 1.551, 0.900, and 1, respectively.

An example of relative response factor values observed for impurities of Formulas I-VII and desvenlafaxine is 0.87, 1.01, 1.10, 1.33, 0.87, 1.13, 1.0, and 1.0, respectively.

In embodiments, the application provides stabilized pharmaceutical formulations containing desvenlafaxine, wherein the total drug-related impurities are less than about 0.5% of the label content of desvenlafaxine.

In embodiments, the application includes methods of preparing stabilized desvenlafaxine formulations, comprising:

a) preparing a solid dispersion or premix composition containing desvenlafaxine;

b) mixing the formed composition with at least one pharmaceutically acceptable excipient; and

c) formulating the mixture into a suitable dosage form.

In embodiments, the application includes methods of preparing stabilized desvenlafaxine formulations, comprising:

a) preparing a solid dispersion or premix composition containing desvenlafaxine;

b) mixing the composition with at least one release controlling substance;

c) optionally, mixing the composition of b) with one or more additional pharmaceutically acceptable excipients; and

d) formulating the mixture into a suitable dosage form.

In embodiments, the application includes methods of preparing stabilized desvenlafaxine formulations, comprising:

a) preparing a solid dispersion or premix composition containing desvenlafaxine;

b) mixing the composition with at least one release controlling substance;

c) mixing the composition of b) with one or more additional pharmaceutically acceptable excipients;

d) formulating the mixture into a tablet, and

e) optionally, coating the tablet.

In embodiments, the application relates to stabilized modified release formulations of desvenlafaxine, wherein concentrations of the active agent desvenlafaxine are in the range of about 0.1% to 95%, or about 3% to about 30%, or about 50% to about 80%, by weight of a total formulation.

In embodiments, the application relates to stabilized modified release formulations of desvenlafaxine, wherein concentrations of a release controlling substance are in the range of about 0.1% to 95%, or about 5% to about 40%, or about 70% to about 95%, by weight of a total formulation.

In embodiments, a rate controlling substance useful in making a modified release formulation of desvenlafaxine includes, but is not limited to: a hydrophilic substance; a hydrophobic substance; a lipophilic substance; a polymer such as a pH dependent polymer, pH independent polymer, swelling polymer, non-swelling polymer, gelling polymer, non-gelling polymer, water soluble polymer, water insoluble polymer; a gum; a wax; an oily substances; Gelucire® products (polyethylene glycol glycerides composed of mono-, di- and tri-glycerides and mono- and diesters of polyethylene glycol); a hydrogenated vegetable oil; alginic acid or an alginate; an acrylic and/or methacrylic acid polymer or copolymer; and the like, including any combinations thereof.

Specific examples of useful polymers include, without limitation thereto: cellulose ethers, e.g., hydroxypropyl methylcelluloses (hypromelloses or HPMC), hydroxypropyl celluloses (HPC), hydroxyethyl celluloses (HEC), ethylcelluloses, and carboxymethylcellulose sodium; polyvinylpyrrolidones, including non-crosslinked polyvinylpyrrolidones; carboxymethylstarches; polyethylene glycols; polyoxyethylenes; poloxamers (polyoxyethylene-polyoxypropylene copolymers); polyvinylalcohols; glucanes (glucans); carrageenans; scleroglucanes (scleroglucans); mannans; galactomannans; gellans; alginic acid and derivatives (e.g., sodium or calcium alginate, propylene glycol alginate); polyaminoacids (e.g. gelatin); methyl vinyl ether/maleic anhydride copolymers; polysaccharides (e.g. carageenan, guar gum, xanthan gum, tragacanth and ceratonia); alpha-, beta- or gamma-cyclodextrins; dextrin derivatives (e.g. dextrin); polymethacrylates (e.g. copolymers of acrylic and methacrylic acid esters containing quaternary ammonium groups); acrylic acid polymers (e.g., carbomers); shellac and derivatives thereof; cellulose acetates; cellulose butyrates; cellulose diacetates; cellulose triacetates; cellulose propionates; cellulose acetate butyrates and other acetylated cellulose derivatives; and the like, including any mixtures of two or more thereof.

Examples of lipophilic/hydrophobic substances that can be used in the present application include, without limitation thereto, waxes (e.g., carnauba wax, microcrystalline wax, beeswax, and polyethoxylated beeswax), natural fats (coconut, soya, cocoa) including modified forms such as totally or partially hydrogenated, hydrogenated castor oil, hydrogenated vegetable oil, and fatty acid derivatives such as mono-, bi- and tri-substituted glycerides, phospholipids, glycerophospholipids, glyceryl palmitostearate, glyceryl behenate, glyceryl monostearate, diethyleneglycol palmitostearate, polyethyleneglycol stearate, polyethyleneglycol palmitostearate, polyoxyethylene-glycol palmitostearate, glyceryl monopalmitostearate, cetyl palmitate, fatty alcohols associated with polyethoxylate fatty alcohols, cetyl alcohol, stearic acid, saturated or unsaturated fatty acids and their hydrogenated derivatives, lecithin, cephalins, chitosan and derivatives thereof, sphingolipids, sterols such as cholesterol and its substituted derivatives, etc.

Useful pH independent polymers according to the present application include, but are not limited to, carbomers, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidones, polyvinyl acetates, mixtures of polyvinyl acetate and polyvinylpyrrolidone polymers, polyvinyl alcohols, polyglycolides, polysiloxanes, polyurethanes and copolymers thereof, alkyl celluloses, hydroxyalkyl celluloses, cellulose ethers, cellulose esters, nitrocelluloses, methyl celluloses, ethyl celluloses, hydroxypropyl celluloses, hydroxypropyl methylcelluloses, hydroxybutyl methylcelluloses, natural polymers such as alginates and other polysaccharides that include but are not limited to arabinans, fructans, fucans, galactans, galacturonans, glucans, mannans, xylans (such as, for example, inulin), levan, fucoidan, carrageenan, galatocarolose, pectic acid, pectin, amylose, pullulan, glycogen, amylopectin, cellulose, dextran, pustulan, chitin, agarose, keratan, chondroitan, dermatan, hyaluronic acid, alginic acid, xanthan gum, starch and various other natural homopolymer or heteropolymers such as those containing one or more of the following viz. aldoses, ketoses, acids or amines, erythrose, threose, ribose, arabinose, xylose, lyxose, allose, altrose, glucose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof, and including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, synthetic polymers such as polymers of lactic acid and glycolic acid, polyanhydrides, poly(ortho) esters, polyurethanes, poly(butyric acid), poly(valeric acid), poly(caprolactone), poly(hydroxybutyrate), poly(lactide-co-glycolide) and poly (lactide-co-caprolactone) copolymers, and mixtures thereof.

Kollidon® SR, sold by BASF, is a spray formulated, free flowing powder composed of about 80% polyvinyl acetate, about 19% polyvinylpyrrolidone, about 0.8% sodium lauryl sulfate, and about 0.2% silica, all percentages expressed by weight. The polyvinyl acetate component has an average molecular weight of 450,000 and the polyvinylpyrrolidone component has an average molecular weight of 44,000-54,000 (i.e., that of Kollidon 30).

Various pH dependent polymers according to the present application include, but are not limited to, Eudragit® 100, Eudragit RS PO and RL PO, Eudragit ND 40, polymers and copolymers of acrylic and methacrylic acids, cellulose acetate butyrate, cellulose acetate phthalate, hydroxypropyl methyl cellulose phthalate, poly(methyl methacrylate), poly(ethylmethacrylate), poly(butylmethacrylate), poly (isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), and any mixtures thereof.

In embodiments, a modified release formulation of desvenlafaxine includes Eudragit® RS PO, Eudragit® RL PO, Kollidon® SR, or combinations thereof as a rate controlling polymer. In embodiments, Eudragit® RS PO and Eudragit® RL PO are used in weight ratios such that 1 part of Eudragit® RL PO is used together with 0-10 parts of Eudragit® RS PO. In embodiments, Eudragit® RS PO and/or Eudragit® RL PO, and Kollidon® SR are used in weight ratios such that 1 part Eudragit® product is used together with 0.1 to 10 parts of Kollidon® SR.

In embodiments, Eudragit® RSPO, and/or Eudragit® RLPO, and desvenlafaxine are used in weight ratios such that 1 part Eudragit® product is used together with 1 to 10 parts of desvenlafaxine.

In embodiments, Kollidon® SR and Eudragit® RS PO, and/or Eudragit®RLPO, are used in weight ratios such that 1 part Kollidon® SR is used together with 0 to 10 parts of Eudragit®RSPO and/or Eudragit® RL PO.

Eudragit® polymers are products of Evonik Industries AG, Essen, Germany. Commercially available products include, but are not limited to, Eudragit RL, Eudragit RS, Eudragit RLPO, Eudragit RSPO, Eudragit RD, Eudragit L, Eudragit S, Eudragit L 100-5, Eudragit NE 30 D and NE 40 D, and Eudragit E 100.

The polymers sold as Eudragit have the general repeating unit:

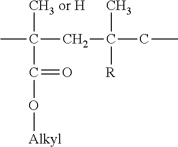

where: R is COOH for the Eudragit L products; R is COOCH₂N(CH₃)₂ for the Eudragit E products; R is COOCH₃ for the Eudragit NE 30 D product and COOC₄H₉ for the NE 40 D; and R is COOCH₂CH₂N⁺(CH₃)₃C1″ for the Eudragit RL and RS products. The alkyl groups vary between different products, and have 1-4 carbons.

The United States Pharmacopoeia and National Formulary describes “methacrylic acid copolymer” as a fully polymerized copolymer of methacrylic acid and an acrylic or methacrylic ester. Three types of copolymers, namely Type A, Type B, and Type C, are defined in the monograph. They vary in their methacrylic acid content and solution viscosity. Type C may contain suitable surface-active agents. The polymers, Type A (e.g., Eudragit L) and Type B (e.g., Eudragit S), can be referred to as “methacrylate copolymers,” consisting of fully polymerized copolymers of acrylic and methacrylic acid esters with a low content of quaternary ammonium groups.

Useful water soluble or insoluble polymers include, for example, sugars, zein, hydroxypropyl celluloses, hydroxypropyl methylcelluloses, hydroxyethyl celluloses, polyvinyl alcohols, polyethylene glycols (PEG), poloxamers (e.g., Pluronic™ products), ethyl celluloses, gelatin, polyarginines, polyglycines, polyvinylpyrrolidones, vinyl acetate copolymers, and any mixtures thereof.

Enteric coating polymers that can be used, for example, include cellulose acetate phthalates (CAP), hydroxypropyl methylcellulose phthalates (HPMCP), polyvinyl acetate phthalates (PVAP), hydroxypropyl methylcellulose acetate succinates (HPMCAS), cellulose acetate trimellitates, hydroxypropyl methylcellulose succinates, cellulose acetate succinates, cellulose acetate hexahydrophthalates, cellulose propionate phthalates, copolymers of methylmethacrylic acid and methyl methacrylate, copolymers of methyl acrylate, methylmethacrylate and methacrylic acid, copolymers of methylvinyl ether and maleic anhydride (e.g., Gantrez™ ES series), ethyl methyacrylate-methylmethacrylate-chlorotrimethylammonium ethyl acrylate copolymers, natural resins such as zein, shellac, and copal collophorium, carboxymethyl ethylcelluloses, co-polymerized methacrylic acid/methacrylic acid methyl esters such as, for instance, materials known under the trade name Eudragit® L12.5, L100, or Eudragit® S12.5, S100, and several commercially available enteric dispersion systems (e.g., Eudragit® L30D55, Eudragit® FS30D, Eudragit® L100-55, Eudragit® S100, Kollicoat® MAE30D and 30DP (BASF), Estacryl® 30D (Eastman Chemical), Aquateric® and Aquacoat® CPD30 (FMC), and any mixtures thereof.

In embodiments, the application includes stabilized compositions and/or formulations, wherein desvenlafaxine retains its physical form during stability testing. In some testing procedures, samples are stored unpackaged or in closed containers under “accelerated” stability testing conditions such as 30° C. and 60% relative humidity (RH), 40° C. and 75% RH, or 60° C., for desired times, such as about one, three, six, or twelve months. Impurities, drug concentrations, and other parameters can be measured at intervals during the storage.

Desvenlafaxine is sensitive to moisture and tends to degrade in the presence of water. Therefore, in order to produce a stabilized pharmaceutical formulation, non-aqueous processes, i.e. direct compression, dry granulation, or dry powder layering techniques are frequently used. Further, non-aqueous granulation methods containing one or more organic solvents can also be used, which prevent exposure of drug particles to moisture, thus imparting polymorphic stability to the formulation.

In embodiments, humidity conditions for the processing area are controlled. In embodiments, the processing is conducted below 20% RH at 25° C. Desvenlafaxine succinate-PVP premixes are hygroscopic and pick up moisture rapidly under ambient room temperature conditions; thus, in normal RH conditions, processing becomes difficult. Further, low moisture content is also intended to impart improved polymorphic stability to the pharmaceutical formulations. Excipients having moisture content less than about 6%, or less than about 2%, or less than about 1%, can additionally be used to minimize polymorphic conversions.

Desvenlafaxine-containing compositions of the application can be further processed into various pharmaceutical dosage forms as prepared, or can be combined with one or more pharmaceutically acceptable excipients. The different pharmaceutical dosage forms comprising compositions of the present application include solid oral dosage forms such as, but not limited to, powders, granules, pellets, tablets, and capsules. Modified release compositions may comprise hydrophilic, lipophilic, or hydrophobic release controlling substances, or their combinations to form matrix or reservoir, or combinations of matrix and reservoir, systems. Compositions may be prepared by extrusion and spheronization, or by using melt granulation techniques. Formulations may be presented as uncoated, film coated, sugar coated, compression-coated, powder coated, enteric coated or modified release coated forms.

In embodiments, pharmaceutical formulations comprising desvenlafaxine may further contain one or more additional active agents such as clovoxamine, femoxetine, flesinoxan, citalopram, escitalopram, fluoxetine, fluvoxamine, paroxetine, sertraline, duloxetine, mirtazapine, venlafaxine, atomoxetine, reboxetine, thionisoxetine, bupropion, mianserin, nefazodone, trazodone, doxepin, amitriptyline, amoxapine, clomipramine, desipramine, doxepin, imipramine, maprotiline, nortriptyline, protriptyline, trimipramine, tranylcypromine, isocarboxazid, phenelzine, selegiline, moclobemide, buspirone, tryptophan, pindolol, agomelatine, amibegron, casopitant, delucemine, elzasonan, gepirone, mecamylamine, milnacipran, miraxion, nemifitide, pexacerfont, saredutant, tofisopam, vestipitant, vilazodone, aripiprazole, clozapine, loxapine, olanzapine, paliperidone, quetiapine, risperidone, sertindole, ziprasidone, asenapine, iloperidonepimavanserin, lithium, valproic acid, carbamazepine, eslicarbazepine, lamotrigine, levetiracetam, oxcarbazepine, tiagabine, topiramate, vigabatrin, zonisamide, riluzole, varenicline, including pharmaceutically active salts, esters, or prodrugs thereof.

Formulations of the present application may include any one or more of other pharmaceutically acceptable excipients such as surfactants, disintegrants, stabilizers, pH dependent or pH independent polymers, and binders. Cores may be prepared by homogenously mixing desvenlafaxine and one or more pharmaceutically acceptable excipients such as those mentioned hereinabove. In embodiments, cores of the present application comprise inert materials such as a diluent or sugar spheres, onto which a fluid or powder containing desvenlafaxine is sprayed or layered. The mixture is then formulated into small beads, pellets, granules, fine granules, or mini-tablets, and filled into hard gelatin or soft gelatin capsules, or compressed into tablets, using conventional procedures.

In embodiments, formulations of the present application are in the form of film-coated tablets. Useful coating compositions comprise conventional film-coating mixtures such as Opadry® products (sold by Colorcon), or other hydrophilic or hydrophobic substances, or mixtures thereof. Other useful additives for coating include, but are not limited to, plasticizers, antiadherents, opacifiers, solvents, and optionally colorants, lubricants, pigments, antifoam agents, and polishing agents.

Plasticizers include materials such as polyethylene glycols (PEG), propylene glycols, cetanol, triacetin, citric acid esters such as, for instance, those sold under the trade name Citroflex® (Pfizer, N.Y.), phthalic acid esters, dibutyl succinate, castor oil, diacetylated monoglycerides, dibutyl sebacate, diethyl phthalate, glycerin, triethyl citrate, and the like. Pigments, opacifiers like titanium dioxide, talc, and other additives may also be included into a coating composition. The quantities of the coatings of the present application may vary from about 0.1% to 10%, or about 0.5 to 5%, of the total weight of a core composition. In an embodiment, a coating is applied either directly onto the core or onto subcoated cores using conventional coating techniques such as, for instance, pan coating or fluidized bed coating methods.

Antiadhesives are frequently used in a film coating process to avoid sticking effects during film formation and drying. An example of a useful antiadhesive for this purpose is talc. The antiadhesive is frequently present in a film coating in amounts of about 0.5% (w/w) to 15% (w/w), based upon the total weight of the coating.

In embodiments, tablet formulations of the present application comprise a sub-coating, onto which a film coating is applied.

In embodiments, pharmaceutical formulations of the present application comprise one or more water soluble or hydrophilic excipients, which include pharmaceutically acceptable water soluble or hydrophilic polymers such as, but not limited to, polyvinylpyrrolidones or povidones (such as grades K25, K29, K30, and K90), hydroxypropyl celluloses, hydroxyethyl celluloses, hydroxypropyl methylcelluloses, polyvinylalcohols, carboxymethylcellulose sodium, and mixtures thereof. Further water soluble excipients according to the present application include sugar substances, such as those having low hygroscopicity, and include, for example, mannitol, lactose, fructose, sorbitol, xylitol, maltodextrin, dextrates, dextrins, lactitol, and mixtures of any two or more thereof.

In embodiments, formulations of the present application optionally contain a dissolution enhancer. Dissolution enhancers increase the rate of dissolution of the drug from the carrier. In general, dissolution enhancers are amphiphilic compounds and are generally more hydrophilic than the carrier. Exemplary dissolution enhancers include: salts such as sodium chloride, potassium chloride, lithium chloride, calcium chloride, magnesium chloride, sodium sulfate, potassium sulfate, sodium carbonate, magnesium sulfate, and potassium phosphate; alcohols such as stearyl alcohol, cetyl alcohol, and polyethylene glycol; surfactants, such as poloxamers (such as poloxamer 188, poloxamer 237, poloxamer 338, and poloxamer 407), docusate salts, polyoxyethylene alkyl ethers, polyoxyethylene castor oil derivatives, polysorbates, polyoxyethylene alkyl esters, sodium lauryl sulfate, and sorbitan monoesters; sugars such as glucose, sucrose, xylitol, sorbitol, and maltitol; amino acids such as alanine and glycine; and any mixtures thereof.

In embodiments, the application relates to stabilized modified release formulations of desvenlafaxine, wherein concentrations of a dissolution enhancer are in the range of about 0.1% to 5% by weight of the total formulation.

In embodiments, cores contain one or more release modifying polymers in admixture with desvenlafaxine, to form a matrix. In certain embodiments, a modified release matrix is further coated with a pH dependent polymer or pH independent polymer, or combinations thereof.

In embodiments of the application, desvenlafaxine is used for preparing inclusion complexes with cyclodextrins. In embodiments, an amorphous form of desvenlafaxine is used for preparing inclusion complexes with cyclodextrins. In embodiments, a solid dispersion or premix of desvenlafaxine is used for preparing inclusion complexes with cyclodextrins.

As used herein, “cyclodextrin” refers to any of the natural cyclodextrins, α-cyclodextrin, β-cyclodextrin, and γ-cyclodextrin, and their respective derivatives or analogs. Cyclodextrins (sometimes called cycloamyloses) make up a family of cyclic oligosaccharides, composed of 5 or more α-D-glucopyranoside units linked 1→4, as in amylose (a fragment of starch). The formation of the inclusion compounds greatly modifies the physical and chemical properties of the guest molecules (such as desvenlafaxine in the present application), mostly in terms of water/aqueous solubility. An inclusion complex of desvenlafaxine with cyclodextrins also aids in penetration of the drug into body tissues.

Any cyclodextrin, which enhances the aqueous solubility and/or provides for effective delivery of desvenlafaxine, may be used in the present application. The cyclodextrins of the present application can include the natural occurring cyclodextrins and their derivatives. The natural cyclodextrins include α-cyclodextrin, β-cyclodextrin and γ-cyclodextrin. Derivatives are typically prepared by modifying the hydroxyl groups located on the exterior or hydrophilic side of the cyclodextrin. The modifications can be made to increase the aqueous solubility and the stability of the complexes and can modify the physical characteristics of the complexes, including the formation and dissociation of the complex. The types and degrees of modification, as well as their preparation, are well-known in the art.

Any of the natural cyclodextrins can be derivatized, such as derivatives of β-cyclodextrin. Cyclodextrin derivatives include alkylated cyclodextrins, such as forming methyl-, dimethyl-, trimethyl- and ethyl-β-cyclodextrins; hydroxyalkylated cyclodextrins, including hydroxyethyl-, hydroxypropyl-, and dihydroxypropyl-β-cyclodextrin; ethylcarboxymethyl cyclodextrins; sulfate, sulfonate and sulfoalkyl cyclodextrins, such as β-cyclodextrin sulfate, β-cyclodextrin sulfonate, and β-cyclodextrin sulfobutyl ether; as well as polymeric cyclodextrins. Other cyclodextrin derivatives can be made by substitution of the hydroxy groups with saccharides, such as glucosyl- and maltosyl-β-cyclodextrin.

Other useful cyclodextrins include the naturally occurring cyclodextrins, methyl-β-cyclodextrin, dimethyl-β-cyclodextrin, trimethyl-β-cyclodextrin, 2-hydroxymethyl-β-cyclodextrin, hydroxyethyl-β-cyclodextrin, 2-hydroxypropyl-β-cyclodextrin, 3-hydroxypropyl-β-cyclodextrin, β-cyclodextrin sulfate, β-cyclodextrin sulfonate, and β-cyclodextrin sulfobutyl ether. Any of the above cyclodextrins or their derivatives or polymers prepared from them can be used for preparation of compositions of the application, either alone or in the form of mixtures of one or more cyclodextrins.

In embodiments, pharmaceutical compositions of the present application comprise desvenlafaxine, adsorbed onto at least one pharmaceutically acceptable carrier. Useful carriers according to the present application include, but are not limited to, polyvinylpyrrolidones, hydroxypropyl methylcelluloses, sugar substances such as mannitol, sorbitol, and the like.

In embodiments, the application includes modified release pharmaceutical formulations comprising desvenlafaxine, optionally together with one or more pharmaceutically acceptable excipients, wherein the formulations are in multi-particulate form.

In embodiments, the application includes modified release pharmaceutical formulations comprising cores containing desvenlafaxine, optionally together with one or more pharmaceutically acceptable excipients, having a coating comprising one or more polymers, wherein the formulations are in multi-particulate form.

In embodiments, modified release multi-particulates of desvenlafaxine comprise non-pariel cores such as pharmacologically inert sugar or similar substances, upon which desvenlafaxine is loaded, optionally together with one or more pharmaceutically acceptable excipients, using any techniques such as powder layering, solution spraying, suspension spraying, or any other techniques known to the art.

In embodiments, modified release formulations of the application comprise desvenlafaxine-loaded non-pariel cores, having a coating comprising one or more pH independent polymers, pH dependent polymers, or combinations thereof.

In embodiments, the application includes pharmaceutical formulations comprising modified release multi-particulates, comprising desvenlafaxine-containing cores and a coating comprising one or more polymers, and optionally having one or more further coatings.

In embodiments, multi-particulates comprising desvenlafaxine further contain one or more non-functional or functional coatings, to provide modified release of the active agent.

Multi-particulate formulations of the application can be prepared using techniques described herein, as well as other methods known to those having skill in the art.

In embodiments, multi-particulates comprising desvenlafaxine are coated with different concentrations of polymers, giving portions having different release profiles, and these can be combined to form a pharmaceutical formulation or dosage form to achieve desired modified release profiles.

In embodiments, multi-particulates comprising desvenlafaxine are coated with different types of polymers, either enteric polymers (pH dependent polymers) or modified release polymers (pH independent polymers) giving different release profiles, and these can be combined to form a pharmaceutical composition or dosage form to achieve desired modified release profiles.

In embodiments, multi-particulates comprising desvenlafaxine can be combined with pharmaceutically acceptable excipients, and compounded to form a pharmaceutical formulation, which can be compressed into tablets or placed into suitable capsule shells, using techniques known to those having skill in the art. In embodiments, compositions of the present application are filled into a hard gelatin capsules, wherein empty hard gelatin capsule shells comprise one or more of hydroxypropylmethyl cellulose, carrageenan, potassium chloride, polyvinyl polymers such as polyvinyl acetate and polyvinyl alcohol, and the like.

Pharmaceutically acceptable excipients according to the present application include, for example, any one or more of diluents, binders, stabilizers, lubricants, glidants, disintegrating agents, anti-oxidants, surfactants, colorants, and other additives that are commonly used in solid pharmaceutical dosage form preparations.

Various useful fillers or diluents according to the present application include, but are not limited to, starches, lactose, cellulose derivatives, confectioner's sugar and the like. Different grades of lactose include, but are not limited to, lactose monohydrate, lactose DT (direct tableting), lactose anhydrous, Flowlac™ (available from Meggle Products), Pharmatose™ (available from DMV), and others. Different starches include, but are not limited to, maize starch, potato starch, rice starch, wheat starch, pregelatinized starch (commercially available as PCS PC10 from Signet Chemical Corporation) and starch 1500, starch 1500 LM grade (low moisture content grade) from Colorcon, fully pregelatinized starch (commercially available as National 78-1551 from Essex Grain Products), and others. Different cellulose compounds that can be used include crystalline cellulose and powdered cellulose. Examples of crystalline cellulose products include, but are not limited to, Ceolus™ KG801, Avicel™ PH101, PH102, PH301, PH302 and PH-F20, PH-112, microcrystalline cellulose 114, and microcrystalline cellulose 112. Other useful diluents include, but are not limited to, carmellose, sugar alcohols such as mannitol (e.g., Pearlitol™ SD200), sorbitol, and xylitol, calcium carbonate, magnesium carbonate, dibasic calcium phosphate, and tribasic calcium phosphate.

Various useful binders according to the present application include, but are not limited to, hydroxypropylcelluloses, also called HPC (e.g., Klucel™ LF, Klucel™ EXF) and useful in various grades, hydroxypropyl methylcelluloses, also called hypromelloses or HPMC (e.g., Methocel™ products) and useful in various grades, polyvinylpyrrolidones or povidones (such as grades K25, K29, K30, and K90), copovidones (e.g., Plasdone™ S 630), powdered acacia, gelatin, guar gum, carbomers (e.g., Carbopol® products), methylcelluloses, polymethacrylates, and starches.

Various useful disintegrants include, but are not limited to, carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™ from FMC-Asahi Chemical Industry Co., Ltd.), crospovidones, examples of commercially available crospovidone products including but not limited to crosslinked povidones, Kollidon® CL from BASF (Germany), Polyplasdone™ XL, XI-10, and INF-10 from ISP Inc. (USA), and low-substituted hydroxypropylcelluloses. Examples of low-substituted hydroxypropylcelluloses include, but are not limited to, low-substituted hydroxypropylcellulose LH11, LH21, LH31, LH22, LH32, LH2O, LH30, LH32 and LH33 (all manufactured by Shin-Etsu Chemical Co., Ltd.). Other useful disintegrants include sodium starch glycolate, colloidal silicon dioxide, and starches.

Useful surface-active agents according to the present application include non-ionic, cationic, anionic, and zwitterionic surface-active agents. Useful non-ionic surface-active agents include ethylene glycol stearates, propylene glycol stearates, diethylene glycol stearates, glycerol stearates, sorbitan esters (e.g., Span™ products) and polyhydroxyethylenically treated sorbitan esters (e.g., Tween™ products), aliphatic alcohols and PEG ethers, phenol and PEG ethers. Useful cationic surface-active agents include quaternary ammonium salts (e.g., cetyltrimethylammonium bromide) and amine salts (e.g., octadecylamine hydrochloride). Useful anionic surface-active agents include sodium stearate, potassium stearate, ammonium stearate, and calcium stearate, triethenolamine stearate, sodium lauryl sulphate, sodium dioctylsulphosuccinate, and sodium dodecylbenzenesulphonate. Natural surface-active agents may also be used, such as for example phospholipids, e.g. diacylphosphatidyl glycerols, diaceylphosphatidyl cholines, and diaceylphosphatidic acids, the precursors and derivatives thereof, such as for example soybean lecithin and egg yolk.

In embodiments, stabilized compositions of the present application contain at least one antioxidant. The antioxidant may be present either as a part of the composition or a packaging component. Thus, in one particular embodiment, antioxidants are introduced into the formulation during the drug loading stage over the inert cores. The antioxidants are present in amounts effective to retard decomposition of desvenlafaxine, as it is susceptible to oxidation.

In embodiments, the content of antioxidant in the formulations ranges from about 0.001 to 10 weight percent, with respect to the active agent content.

Among the antioxidants, non-limiting examples that may be mentioned include ascorbic acid and its salts, tocopherols, sulfite salts such as sodium metabisulfite or sodium sulfite, sodium sulfide, dl-alpha-tocopherol, butylated hydroxyanisole, butylated hydroxytoluene, ascorbyl palmitate, and propyl gallate. Other suitable antioxidants will be readily recognized by those skilled in the art. In embodiments, sodium metabisulfite, sodium sulfite, sodium sulfide, or any mixtures thereof, are useful as antioxidants.

Useful lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and any combinations thereof.

One or more glidant materials, which improve the flow of powder blends, pellets or mini-tablets and minimize dosage form weight variations, can be used. Useful glidants include, but are not limited to, silicon dioxide, talc, and combinations thereof.

Coloring agents can be used to color code the compositions, for example, to indicate the type and dosage of the therapeutic agent therein. Coloring agents can also be used to differentiate the varied fractions of multi-particulates comprised in a unit dosage form such as a capsule. Suitable coloring agents include, without limitation, natural and/or artificial compounds such as FD&C coloring agents, natural juice concentrates, pigments such as titanium oxide, silicon dioxide, iron oxides, zinc oxide, any combinations thereof, and the like.

Various solvents that can be used in processes of preparation of pharmaceutical formulations of the present application include, but are not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulfoxide, N,N-dimethylformamide, tetrahydrofuran, and any mixtures thereof.

In embodiments, one or more pH independent or pH dependent polymers are used for coating the compositions of the present application.

The descriptions of excipients are illustrative and are not intended to be exhaustive. Those skilled in the art will be aware of many other substances that are useful in the practice of the application, and the use of such substances is specifically contemplated in this application.

In an aspect, the application provides methods of preparing pharmaceutical compositions comprising desvenlafaxine.

An aspect provides processes for preparing solid dispersion or premix compositions of the present application, embodiments comprising:

a) intimately mixing desvenlafaxine with at least one pharmaceutically acceptable excipient such as a hydrophilic polymer; and

b) obtaining a solid dispersion or premix composition of desvenlafaxine.

An aspect provides processes for preparing solid dispersion or premix compositions of the present application, embodiments comprising:

a) dissolving desvenlafaxine and at least one pharmaceutically acceptable excipient, such as a hydrophilic polymer, in a suitable solvent; and

b) removing the solvent.

An aspect provides processes for preparing formulations of the present application, embodiments comprising:

a) preparing a solid dispersion or premix composition of desvenlafaxine;

b) mixing the composition with at least one excipient; and

c) formulating the mixture into a suitable dosage form.

In an aspect, the application provides processes for preparing formulations of the present application, embodiments comprising:

a) preparing a solid dispersion or premix composition of desvenlafaxine;

b) mixing the composition with at least one release controlling substance;

c) optionally, further mixing the composition of b) with one or more additional pharmaceutically acceptable excipients; and

d) formulating the mixture into a suitable dosage form.

In an aspect, the application provides processes for preparing formulations of the present application, embodiments comprising:

a) preparing a solid dispersion or premix composition of desvenlafaxine;

b) mixing the composition with at least one release controlling substance;

c) further mixing the composition of b) with one or more other pharmaceutically acceptable excipients;

d) formulating the mixture into a tablet, and

e) coating the tablet.

In embodiments, pharmaceutical formulations of desvenlafaxine are prepared utilizing a direct compression, dry granulation, or wet granulation method.

Equipment suitable for processing the pharmaceutical formulations of the present application include any one or more of rapid mixer granulators, planetary mixers, mass mixers, ribbon mixers, fluid bed processors, mechanical sifters, blenders, roller compacters, extrusion-spheronizers, compression machines, capsule filling machines, rotating bowls or coating pans, tray dryers, fluid bed dryers, rotary cone vacuum dryers, and the like, multimills, fluid energy mills, ball mills, colloid mills, roller mills, hammer mills, and the like, equipped with a suitable screen.

In embodiments, the application includes stabilized pharmaceutical compositions of desvenlafaxine, which may be prepared by spray drying a suspension or solution of desvenlafaxine and a water soluble sugar derivative, with or without an organic base, optionally together with one or more pharmaceutically acceptable excipients. Alternatively, desvenlafaxine compositions may also be prepared by fluid bed granulation techniques, where a solution of desvenlafaxine, optionally together with one or more pharmaceutically acceptable excipients, is sprayed onto inert cores or layered onto inert cores.

In embodiments, desvenlafaxine-containing compositions may be prepared by powder layering techniques, wherein a drug layering powder comprising desvenlafaxine, together with one or more pharmaceutically acceptable excipients, is layered onto inert cores while being sprayed with a binder solution.

In embodiments, the application includes packaging for desvenlafaxine-containing formulations that are stable during storage and/or transportation. Stabilization can be improved by using package forms such as packages suppressing the permeation of oxygen and moisture, packages having air replaced with an inert gas (i.e., gases that do not contain oxygen), vacuum packages, and packages including a deoxidizer. The stabilization is improved by reducing oxygen amounts, with which the solid preparation is directly brought in contact, using such package forms. When a deoxidizer is used, the pharmaceutical solid preparation can be surrounded by an oxygen permeating material and placed inside other packaging containing the deoxidizer.

In embodiments, stabilized compositions of the present application include a desiccant and/or an oxygen adsorbent as a component of packaging. A desiccant is a hygroscopic substance that induces or sustains a state of dryness (desiccation) in its local vicinity in a moderately well-sealed container. Commonly used pre-packaged desiccants are solids, and work through absorption or adsorption of water, or a combination of the two. Desiccants for specialized purposes may be in forms other than solid, and may work through other principles, such as chemical bonding of water molecules. Pre-packaged desiccant is most commonly used to remove excessive humidity that would normally degrade or even destroy products sensitive to moisture. Non-limiting examples of various desiccants are anhydrous calcium sulfate (e.g., Drierite® products), silica gel, calcium sulfate, calcium chloride, montmorillonite clay, and molecular sieves. Oxygen adsorbents such as StabilOx® are useful in minimizing the degradation of active agent due to oxidation.

In an aspect, the application also provides methods of prophylaxis, amelioration or treating depression such as major depressive disorder in mammals and man by administering a therapeutically effective amount of a formulation to a subject in need thereof.

The dosage forms can be subjected to in-vitro dissolution testing, such as according to Test 711 “Dissolution” in United States Pharmacopeia 29, United States Pharmacopeial Convention, Inc., Rockville, Md., 2005 (USP), to determine the rate at which an active ingredient is released from the dosage forms, and content of active substance can be determined in dissolution media using techniques such as HPLC.

A swelling study can performed on dosage forms prepared using different polymers in order to predict their in vitro dissolution performance and drug release characteristics. The dosage forms are immersed into an aqueous liquid and the dimensions length and width (or diameter and height for cylindrical shapes) are measured before and at intervals during the test. Dosage forms having similar swelling properties may have comparable drug dissolution profiles.

In embodiments, tablets of the present application are packaged in high density polyethylene (HDPE) bottles with closures, optionally together with a molecular sieve desiccant pouch and/or polyester as a filler.

In embodiments, pharmaceutical dosage forms of the present application are intended for oral, buccal, or sublingual administration to a subject in need thereof.

The following examples further describe certain specific aspects and embodiments of the application. The examples are provided solely for the purpose of illustration, and should not be construed as limiting the scope of the disclosure in any manner.

### Example 1

### Preparation of Desvenlafaxine Premix

| Ingredient | Grams |
|---|---|
| Desvenlafaxine | 100 |
| Succinic acid | 46 |
| Povidone K30 | 146 |
| Sodium metabisulfite | 0.4 |
| Methanol* | q.s. |

*Evaporates during processing.

Manufacturing Procedure:

1. Desvenlafaxine is dissolved in methanol at 30±5° C.

2. Succinic acid is added to the solution and the mixture is stirred at 30±5° C.

3. Povidone is dissolved in methanol under a nitrogen atmosphere.

4. Sodium metabisulfite is added to the solution of step 3 and the mixture is stirred at 30±5° C. for 15-30 minutes, then the solution is filtered.

5. The filtrate from step 4 is combined with the solution of step 2 and the mixture is stirred at 30±5° C.

6. The mixture of step 5 is spray-dried at 42.5±7.5° C.

7. Spray-dried material dried in a vacuum tray dryer at 30±5° C. and at least 600 mmHg vacuum for 45±15 minutes, followed by drying at 47.5±2.5° C. and under at least 600 mm Hg vacuum for 18 hours.

8. The dried material of step 7 is sifted through a #20 mesh sieve.

The product contains desvenlafaxine succinate and povidone in a 1:1 weight ratio, and is used as the drug substance for the formulations of all of the following examples.

### Example 2

### Desvenlafaxine Tablets

| Ingredient | mg/Tablet |
|---|---|
| Desvenlafaxine succinate-povidone premix | 289.7 |
| Polyvinyl acetate-povidone, 8:2 by wt. (Kollidon ® SR) | 390 |
| Microcrystalline cellulose | 70 |
| Talc | 5 |
| Magnesium stearate | 1 |

Coating

| | |
|---|---|
| Opadry ® II 85F23452† | 35 |
| Water* | q.s. |

*Evaporates during processing.
†Opadry ® II 85F23452 is a coating product from Colorcon, containing polyvinyl alcohol, PEG 4000, titanium dioxide, talc, and iron oxide red.

Manufacturing Procedure:

1. Sift together desvenlafaxine premix, Kollidon® SR, and microcrystalline cellulose through a #40 mesh sieve, and blend.

2. Sift talc and magnesium stearate through a #60 mesh sieve, add to the mixture of step 1, and blend.

3. Compress the blend of step 3 into tablets.

4. Coat the tablets with an Opadry® aqueous dispersion, and dry.

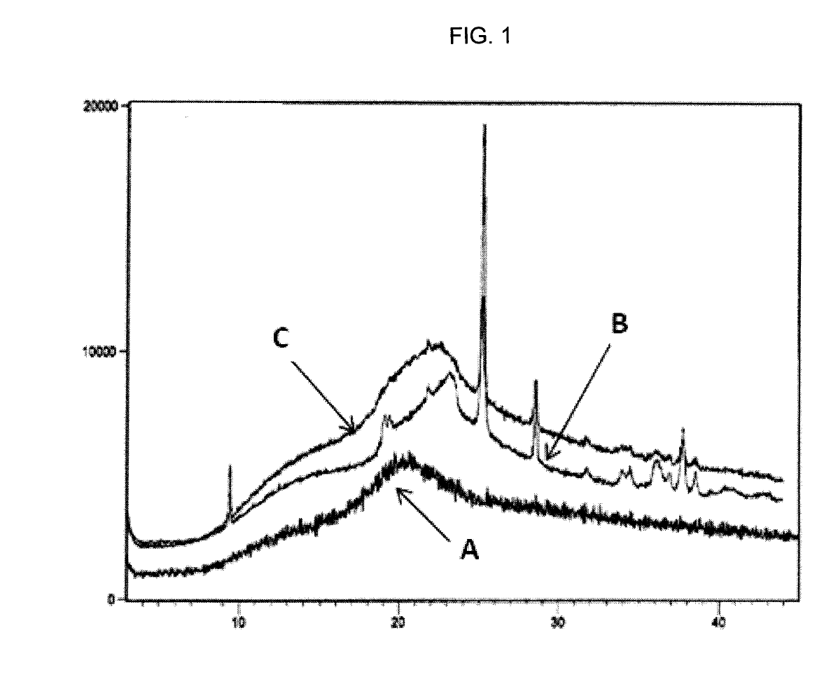

Tablets are analyzed using PXRD. FIG. 1 shows patterns for the desvenlafaxine succinate-PVP premix (“A”), placebo tablets prepared as described, but omitting the desvenlafaxine succinate-PVP premix (“B”), and tablets as prepared (“C”). The amorphous form of the premix is maintained during manufacturing.

### Example 3

### Desvenlafaxine Tablets

| Ingredient | mg/Tablet |
|---|---|
| Desvenlafaxine succinate-povidone premix | 289.7 |
| Kollidon ® SR | 390 |
| Microcrystalline cellulose | 70 |
| Talc | 5 |
| Magnesium stearate | 1 |

Subcoating

| | |
|---|---|
| HPMC 5-6 cP | 11.3 |
| Hydroxypropyl cellulose | 3.7 |
| Dichloromethane* | q.s. |
| Isopropyl alcohol* | q.s. |

Film Coating

| | |
|---|---|
| Opadry ® II 85F23452 | 20 |
| Water* | q.s. |

*Evaporates during processing.

Manufacturing Procedure:

1. Sift together desvenlafaxine premix, Kollidon® SR, and microcrystalline cellulose through a #40 mesh sieve, and blend.

2. Sift talc and magnesium stearate through a #60 mesh sieve, combine with the step 1 mixture, and blend.

3. Compress the blend of step 2 into tablets.

4. Coat the tablets with a mixture of the subcoating materials, using a coating pan, and dry.

5. Coat the tablets of step 4 with an aqueous dispersion of Opadry®, and dry.

Tablets are packaged and stored under the accelerated stability testing conditions of 40° C. and 75% relative humidity (RH) for 3 months. The samples are analyzed for drug content and impurities of Formulas III-V before, during, and after the storage, and results are shown in Table 2, where values are percentages of the label drug content.

TABLE 2

| | | Impurity | | | | |
|---|---|---|---|---|---|---|
| | Drug | Formula | Formula | | Highest | |
| Time | Assay | III | IV | Formula V | Unidentified | Total |
| Initial | 97.8 | 0.05 | 0.01 | ND | 0.01 | 0.12 |
| 1 Month | 99.0 | 0.07 | 0.01 | ND | 0.02 | 0.13 |
| 2 Months | 98.5 | 0.08 | 0.02 | ND | 0.02 | 0.19 |
| 3 Months | 100.2 | 0.08 | 0.04 | ND | 0.05 | 0.23 |

ND = not detected.

### Example 4

### Desvenlafaxine Tablets

| Ingredient | mg/Tablet |
|---|---|
| Desvenlafaxine succinate-povidone premix | 289.7 |
| Eudragit ® RS PO† | 114 |
| Eudragit ® RL PO‡ | 25 |
| Microcrystalline cellulose | 150 |
| Talc | 5 |
| Magnesium stearate | 1 |
| Methanol* | q.s. |

Coating

| | |
|---|---|
| Opadry ® II 85F23452 | 30 |
| Water* | q.s. |

*Evaporates during processing.
†Eudragit ® RS PO is a product of Evonik Industries, categorized as ammonio methacrylate copolymer type B.
‡Eudragit ® RL PO is a product of Evonik Industries, categorized as ammonio methacrylate copolymer type A.

Manufacturing Procedure:

1. Sift together desvenlafaxine premix and microcrystalline cellulose through a #40 mesh sieve, and transfer to a fluid bed processor.

2. Dissolve Eudragit® RSPO and Eudragit® RLPO in methanol.

3. Granulate the blend of step 1 with the step 2 solution, and dry the granules.

4. Sift dried granules of step 3 through a #30 mesh sieve.

5. Sift talc and magnesium stearate through a #60 mesh sieve.

6. Combine materials of steps 4 and 5 and blend.

7. Compress the blend of step 6 into tablets.

8. Coat the tablets of step 7 with an Opadry® aqueous dispersion, and dry.

### Example 5

### Desvenlafaxine Tablets

| Ingredient | mg/Tablet |
|---|---|
| Desvenlafaxine succinate-povidone premix | 289.7 |
| Eudragit ® RS PO | 114 |
| Eudragit ® RL PO | 25 |
| Microcrystalline cellulose | 150 |
| Talc | 5 |
| Magnesium stearate | 1 |
| Methanol* | q.s. |

Subcoating

| | |
|---|---|
| HPMC 5-6 cP | 11.3 |
| Hydroxypropyl cellulose | 3.7 |
| Dichloromethane* | q.s. |
| Isopropyl alcohol* | q.s. |

Film Coating

| | |
|---|---|
| Opadry ® II 85F23452 | 15 |
| Water* | q.s. |

*Evaporates during processing.

Manufacturing Procedure:

1. Sift together desvenlafaxine premix and microcrystalline cellulose through a #40 mesh sieve, and transfer to a fluid bed processor.

2. Dissolve Eudragit® RSPO and Eudragit® RLPO in methanol.

3. Granulate the blend of step 1 with step 2 solution, and dry the granules.

4. Sift dried granules of step 3 through a #30 mesh sieve.

5. Sift talc and magnesium stearate through a #60 mesh sieve.

6. Combine granules of step 4 and step 5 materials, and blend.

7. Compress the blend of step 6 into tablets.

8. Coat the tablets of step 7 with a mixture of subcoating ingredients, using a coating pan, and dry.

9. Coat the tablets of step 8 with an aqueous dispersion of Opadry®, and dry.

### Example 6

### Desvenlafaxine Tablets

| Ingredient | mg/Tablet |
|---|---|
| Desvenlafaxine succinate-povidone premix | 289.7 |
| Hydrogenated castor oil | 150 |
| Microcrystalline cellulose | 150 |
| Talc | 5 |

Coating

| | |
|---|---|
| Opadry ® II 85F23452 | 30 |
| Water* | q.s. |

*Evaporates during processing.

Manufacturing Procedure:

1. Sift together desvenlafaxine premix, hydrogenated castor oil, and microcrystalline cellulose through a #40 mesh sieve, and blend.

2. Sift talc through a #60 mesh sieve and blend with the step 1 mixture.

3. Compress the blend of step 2 into tablets.

4. Coat the tablets of step 3 with an aqueous dispersion of Opadry®, and dry.

### Example 7

### Desvenlafaxine Tablets

| Ingredient | mg/Tablet |
|---|---|
| Desvenlafaxine succinate-povidone premix | 289.7 |
| Hydrogenated castor oil | 150 |
| Microcrystalline cellulose | 150 |
| Talc | 5 |

Subcoating

| | |
|---|---|
| HPMC 5-6 cP | 11.3 |
| Hydroxypropyl cellulose | 3.7 |
| Dichloromethane* | q.s. |
| Isopropyl alcohol* | q.s. |

Film Coating

| | |
|---|---|
| Opadry ® II 85F23452 | 15 |
| Water* | q.s. |

*Evaporates during processing.

Manufacturing Procedure:

1. Sift together desvenlafaxine premix, hydrogenated castor oil, and microcrystalline cellulose through a #40 mesh sieve, and blend.

2. Sift talc through a #60 mesh sieve, combine with the step 1 mixture, and blend.

3. Compress the blend of step 2 into tablets.

4. Coat the tablets of step 3 with a mixture of the subcoating materials, using a coating pan, and dry.

5. Coat the tablets of step 4 with an aqueous dispersion of Opadry®, and dry.

### Example 8

### Desvenlafaxine Tablets

| | mg/Tablet | | |
|---|---|---|---|
| Ingredient | 8A | 8B | 8C |
| Desvenlafaxine succinate-povidone premix | 289.7 | 289.7 | 289.7 |
| Carbopol ® 71G† | 150 | — | 150 |
| Carbopol ® 971P‡ | — | 200 | 200 |
| Microcrystalline cellulose | 100 | 100 | 100 |
| Talc | 5 | 5 | 5 |
| Magnesium stearate | 1 | 1 | 1 |
| Methanol* | — | — | q.s. |

Coating

| | | | |
|---|---|---|---|
| Opadry ® II 85F23452 | 30 | 30 | 30 |
| Water* | q.s. | q.s. | q.s. |

*Evaporates during processing.
†Carbopol ® 71G is a polymer of acrylic acid, crosslinked with allyl sucrose or allyl pentaerythritol, viscosity 4000-11000 cP, in granular form, from The Lubrizol Corporation.
‡Carbopol ® 971P is a polymer of acrylic acid, crosslinked with allyl sucrose or allyl pentaerythritol, viscosity 4000-11000 cP, in powder form, from The Lubrizol Corporation.

Manufacturing Procedures:

### Example 8A

1. Sift together desvenlafaxine premix, Carbopol® 71G, and microcrystalline cellulose through a #40 mesh sieve, and blend.

2. Sift talc and magnesium stearate through a #60 mesh sieve, combine with the step 1 mixture, and blend.

3. Compress the blend of step 2 into tablets.

4. Coat the tablets with an aqueous dispersion of Opadry®, and dry.

### Example 8B

1. Sift together desvenlafaxine premix, Carbopol® 971P, microcrystalline cellulose, and half the quantity of magnesium stearate through a #40 mesh sieve, and blend.

2. Pass the blend of step 1 through a roller compactor.

3. Mill the compacted material and sift through a sieve, collecting the material passing through. Repeat the milling and sifting with material retained on the sieve, until all of the material passes through.

4. Sift talc and the remaining magnesium stearate through a #60 mesh sieve, combine with the material from step 3, and blend.

5. Compress the blend of step 4 into tablets.

6. Coat the tablets with an aqueous dispersion of Opadry®, and dry.

### Example 8C

1. Sift together desvenlafaxine premix, the Carbopol® products, and microcrystalline cellulose through a #40 mesh sieve, and blend.

2. Granulate the mixture of step 1 with methanol, using a fluid bed processor with top spray, and dry the granules.

3. Sift dried granules of step 2 through a #30 mesh sieve.

4. Sift talc and magnesium stearate through a #60 mesh sieve, combine with the granules of step 3, and blend.

5. Compress the blend of step 4 into tablets.

6. Coat the tablets with an aqueous dispersion of Opadry®, and dry.

In-vitro dissolution studies are performed using six tablets of Examples 2, 4, 6, and 8B with the following conditions, and the results are shown in Table 3:

Medium: 0.9% NaCl in water.

Volume: 900 mL.

Apparatus: USP type 1.

Speed: 100 rpm.

Temperature: 37±0.5° C.

TABLE 3

| | Cumulative % of Drug Dissolved | | | | | | | |
|---|---|---|---|---|---|---|---|---|
| | Example 2 | | | | | | | |
| | | % | Example 4 | | Example 6 | | Example 8B | |
| Hours | Avg. | RSD | Avg. | % RSD | Avg. | % RSD | Avg. | % RSD |
| 0 | 0 | 0 | 0 | 0 | 0 | 0 | 0 | 0 |
| 1 | 25 | 4 | 25 | 2 | 20 | 3 | 10 | 24 |
| 2 | 34 | 3 | 35 | 0 | 27 | 0 | 15 | 21 |
| 4 | 48 | 2 | 49 | 0 | 37 | 2 | 21 | 20 |
| 6 | 59 | 3 | 60 | 0 | 44 | 1 | 27 | 17 |
| 8 | 70 | 3 | 68 | 0 | 50 | 1 | 33 | 17 |
| 10 | 80 | 2 | 75 | 0 | 56 | 1 | 37 | 15 |
| 12 | 88 | 1 | 80 | 1 | 60 | 1 | 42 | 15 |
| 16 | 98 | 1 | 88 | 1 | 69 | 1 | 50 | 12 |
| 20 | 100 | 1 | 92 | 1 | 76 | 1 | 58 | 11 |
| 24 | 100 | 1 | 94 | 1 | 82 | 1 | 65 | 10 |

### Example 9

### Desvenlafaxine Tablets

| | mg/Tablet | | |
|---|---|---|---|
| Ingredient | 9A | 9B | 9C |
| Desvenlafaxine succinate-povidone premix | 289.7 | 289.7 | 289.7 |
| Carbopol ® 71G | 150 | — | 150 |
| Carbopol ® 971P | — | 200 | 200 |
| Microcrystalline cellulose | 100 | 100 | 100 |
| Talc | 5 | 5 | 5 |
| Magnesium stearate | 1 | 1 | 1 |
| Methanol* | — | — | q.s. |

Subcoating

| | | | |
|---|---|---|---|
| HPMC 5-6 cP | 11.3 | 11.3 | 11.3 |
| Hydroxypropyl cellulose | 3.7 | 3.7 | 3.7 |
| Dichloromethane* | q.s. | q.s. | q.s. |
| Isopropyl alcohol* | q.s. | q.s. | q.s. |

Coating

| | | | |
|---|---|---|---|
| Opadry ® II 85F23452 | 15 | 15 | 15 |
| Water* | q.s. | q.s. | q.s. |

*Evaporates during processing.

### Manufacturing Procedures

### Example 9A

1. Sift together desvenlafaxine premix, Carbopol® 71 G, and microcrystalline cellulose through a #40 mesh sieve, and blend.

2. Sift talc and magnesium stearate through a #60 mesh sieve, combine with the mixture of step 1, and blend.

3. Compress the blend of step 2 into tablets.

4. Coat the tablets with a mixture of the subcoating ingredients, using a coating pan, and dry.

5. Coat the tablets of step 4 with an aqueous dispersion of Opadry®, and dry.

### Example 9B

1. Sift together desvenlafaxine premix, Carbopol® 971P, microcrystalline cellulose, and half the quantity of magnesium stearate through a #40 mesh sieve, and blend.

2. Pass the blend of step 1 through a roller compactor.

3. Mill the compacted material and sift through a #20 meshsieve, collecting the material passing through. Repeat the milling and sifting with material retained on the sieve, until all of the material passes through.

4. Sift talc and remaining magnesium stearate through a #60 mesh sieve, combine with sifted material of step 3, and blend.

5. Compress the blend of step 4 into tablets.

6. Coat the tablets with a mixture of subcoating ingredients, using a coating pan, and dry.

7. Coat the tablets of step 6 with an aqueous dispersion of Opadry®, and dry.

### Example 9C

1. Sift together desvenlafaxine premix, the Carbopol® products, and microcrystalline cellulose through a #40 mesh sieve, and mix.

2. Granulate the material of step 1 with methanol in a fluid bed processor with top spray, and dry the granules.

3. Sift dried granules through a #30 mesh sieve.

4. Sift talc and magnesium stearate through a #60 mesh sieve, combine with granules of step 3, and blend.

5. Compress the blend of step 4 into tablets.

6. Coat the tablets with a mixture of the subcoating ingredients, using a coating pan, and dry.

Coat the tablets of step 6 with an aqueous dispersion of Opadry®, and dry.

### Example 10

### Desvenlafaxine Tablets

| Ingredient | mg/Tablet |
|---|---|
| Desvenlafaxine succinate-povidone premix | 289.7 |
| Sodium starch glycolate P 5000 | 600 |
| Microcrystalline cellulose | 100 |
| Talc | 5 |
| Magnesium stearate | 1 |
| Methanol* | q.s. |

Coating

| | |
|---|---|
| Opadry ® II 85F23452 | 30 |
| Water* | q.s. |

*Evaporates during processing.

Manufacturing Procedure:

1. Sift together desvenlafaxine premix, sodium starch glycolate, and microcrystalline cellulose through a #40 mesh sieve, and blend.

2. Granulate the blend of step 1 with methanol, and dry the granules.

3. Sift dried granules of step 2 through a #30 mesh sieve.

4. Sift talc and magnesium stearate through a #60 mesh sieve, combine with the granules of step 3, and blend.

5. Compress the blend of step 4 into tablets.

6. Coat the tablets with an aqueous dispersion of Opadry®, and dry.

### Example 11

### Desvenlafaxine Tablets

| Ingredient | mg/Tablet |
|---|---|
| Desvenlafaxine succinate-povidone premix | 289.7 |
| Sodium starch glycolate P 5000 | 600 |
| Microcrystalline cellulose | 100 |
| Talc | 5 |
| Magnesium stearate | 1 |
| Methanol* | q.s. |

Subcoating

| | |
|---|---|
| HPMC 5-6 cP | 11.3 |
| Hydroxypropyl cellulose | 3.7 |
| Dichloromethane* | q.s. |
| Isopropyl alcohol* | q.s. |

Coating

| | |
|---|---|
| Opadry ® II 85F23452 | 15 |
| Water* | q.s. |

*Evaporates during processing.

Manufacturing Procedure:

1. Sift together desvenlafaxine premix, sodium starch glycolate, and microcrystalline cellulose through a #40 mesh sieve.

2. Granulate the blend of step 1 with methanol and dry the granules.

3. Sift dried granules of step 2 through a #30 mesh sieve.

4. Sift talc and magnesium stearate through a #60 mesh sieve, combine with the granules of step 3, and blend.

5. Compress the blend of step 4 into tablets.

6. Coat the tablets with a mixture of the subcoating ingredients, using a coating pan, and dry.

7. Coat the tablets of step 6 with an aqueous dispersion of Opadry®, and dry.

### Example 12

### Desvenlafaxine Tablets

| Ingredient | 12A | 12B | 12C | 12D | 12E | 12F |
|---|---|---|---|---|---|---|
| Desvenlafaxine succinate- | 289.7 | 289.7 | 289.7 | 289.7 | 289.7 | 289.7 |
| povidone premix | | | | | | |
| Kollidon ® SR | — | 100 | 383.8 | — | — | — |
| Eudragit ® RS PO | 114 | 114 | — | — | — | — |
| Eudragit ® RL PO | 25.6 | 25.3 | — | — | — | — |
| Dibasic calcium phosphate | 190 | 80 | — | — | — | — |
| dihydrate | | | | | | |
| Hydrogenated castor oil | — | — | — | 110 | — | — |
| Carbopol ® G | — | — | — | — | 152 | — |
| Carbopol ® 971 | — | — | — | — | — | 195 |
| Microcrystalline cellulose | — | — | — | 200.3 | 34 | — |
| (Avicel ™ PH102) | | | | | | |
| Microcrystalline cellulose | — | — | 70 | — | — | 109 |
| (Avicel ™ PH112) | | | | | | |
| Methanol* | q.s. | q.s. | — | — | — | — |
| Talc | 4 | 4 | 4 | — | 3.5 | 4.2 |
| Magnesium stearate | 2 | 2 | 2.5 | — | 1.7 | 4.2 |

Film Coating

| | | | | | | |
|---|---|---|---|---|---|---|
| Opadry ® II 85F23452 | 15 | 15 | 15 | 15 | 15 | 15 |
| Water* | q.s. | q.s. | q.s. | q.s. | q.s. | q.s. |

*Evaporates during processing.

### Manufacturing Procedures

### Examples 12A and 12B

1. Sift together drug premix and dibasic calcium phosphate through a #40 mesh sieve and transfer to a fluid bed processor.

2. Dissolve Eudragit® RSPO and Eudragit® RLPO in methanol.

3. Granulate the materials of step 1 with the solution of step 2 in the fluid bed processor at 45-50° C., and dry the granules at 50° C.

4. Sift dried granules through a #30 mesh sieve.

5. Sift talc and magnesium stearate through a #60 mesh sieve and sift Kollidon® SR through #40 mesh sieve.

6. Combine materials of step 5 with the granules of step 4 and blend.

7. Compress the blend of step 6 into tablets.

8. Coat the tablets of step 7 with an aqueous dispersion of Opadry®, and dry.

### Example 12C

1. Sift together drug premix, Kollidon® SR and microcrystalline cellulose through a #40 mesh sieve, and blend.

2. Sift talc and magnesium stearate through #60 mesh sieve.

3. Blend the materials of steps 1 and 2.

4. Compress the blend of step 3 into tablets.

### Example 12D

1. Sift together drug premix, hydrogenated castor oil, microcrystalline cellulose through a #40 mesh sieve, and blend.

2. Compress the blend of step 1 into tablets.

### Example 12E

1. Sift together drug premix, Carbopol®, and microcrystalline cellulose through #40 mesh sieve, and blend.

2. Sift talc and magnesium stearate through a #60 mesh sieve.

3. Combine material of steps 2 and 3 and blend.

4. Compress the blend of step 3 to form tablets.

### Example 12F

1. Sift together drug premix, Carbopol®, microcrystalline cellulose, talc, and half the quantity of magnesium stearate through a #40 mesh sieve, and blend.

2. Compact the blend of step 2 to form slugs.

3. Mill the slugs of step 2 and sift through a #24 mesh sieve, collecting the retained material.

4. Repeat steps 2 and 3 using the material passing through the sieve, until a desired quantity of retained material is achieved.

5. Sift the remaining quantity of magnesium stearate through a #60 mesh sieve.

6. Combine material from steps 3 and 4 with material of step 5 and blend.

7. Compress the blend of step 6 into tablets.

8. Coat the tablets using an aqueous dispersion of Opadry®, and dry.

In-vitro dissolution studies are performed using tablets of Examples 12A-12F with the following conditions, and the results are as shown in Table 4:

Medium: 0.9% NaCl in water.

Volume: 900 mL.

Apparatus: USP type 1.

Speed: 100 rpm.

Temperature: 37±0.5° C.

TABLE 4

| | Cumulative % of Drug Dissolved | | | | | |
|---|---|---|---|---|---|---|
| Hours | 12A | 12B | 12C | 12D | 12E | 12F |
| 0 | 0 | 0 | 0 | 0 | 0 | 0 |
| 1 | 31 | 23 | 19 | 29 | 15 | 8 |
| 2 | 42 | 31 | 26 | 42 | 22 | 13 |
| 4 | 58 | 42 | 37 | 61 | 38 | 18 |
| 6 | 69 | 52 | 45 | 76 | 62 | 23 |
| 8 | 80 | 59 | 53 | 89 | 86 | 28 |
| 10 | — | 65 | 61 | 95 | 96 | 32 |
| 12 | 96 | 71 | 69 | 98 | 96 | 37 |
| 16 | 106 | 81 | 83 | 98 | 97 | 44 |
| 20 | — | 89 | 93 | 99 | 100 | 50 |
| 24 | 106 | 97 | 97 | 100 | 100 | 57 |

### Example 13

### Desvenlafaxine Tablets

| Ingredient | 13A | 13B | 13C |
|---|---|---|---|
| Desvenlafaxine succinate-povidone premix | 289.7 | 289.7 | 289.7 |
| Eudragit ® RS PO | 114 | 89 | 114 |
| Eudragit ® RL PO | — | 25.3 | 25.3 |
| Dicalcium phosphate dihydrate | 80 | 190 | 150 |
| Methanol* | q.s. | q.s. | q.s. |
| Talc | 4 | 4 | 4 |
| Magnesium stearate | 2 | 2 | 2 |

Film Coating

| | | | |
|---|---|---|---|
| Opadry ® II 85F23452 | 15 | 15 | 15 |
| Water* | q.s. | q.s. | q.s. |

*Evaporates during processing.

Manufacturing Procedure:

1. Sift together drug premix and dicalcium phosphate dihydrate through a #40 mesh sieve and transfer to a fluid bed processor.

2. Dissolve Eudragit® RSPO and Eudragit® RLPO in methanol.

3. Granulate the blend of step 1 with step 2 solution using fluid bed processor at 50° C., and dry the granules at 50° C.

4. Sift dried granules through a #30 mesh sieve.

5. Sift talc and magnesium stearate through a #60 mesh sieve.

6. Combine the granules of step 4 and the material of step 5 and blend.

7. Compress the blend of step 6 into tablets.

8. Coat the tablets with an aqueous dispersion of Opadry, and dry.

### Example 14

### Desvenlafaxine Tablets

| Ingredient | 14A | 14B | 14C |
|---|---|---|---|
| Desvenlafaxine succinate-povidone premix | 289.7 | 289.7 | 289.7 |
| Kollidon ® SR | — | 383.8 | 383.8 |
| Eudragit ® RS PO | 89 | — | — |
| Eudragit ® RL PO | 25.3 | — | — |
| Dibasic calcium phosphate dihydrate | 190 | 70 | |
| Microcrystalline cellulose (Avicel ™ PH112) | — | — | 70 |
| Methanol* | q.s. | — | — |
| Talc | 4 | 4 | 4 |
| Magnesium stearate | 2 | 2.5 | 2.5 |
| Opadry ® II 85F23452 | 30 | 37.5 | 37.5 |
| Water* | q.s. | q.s. | q.s. |

*Evaporates during processing.

### Manufacturing Procedures

### Example 13A

1. Sift together drug premix and dibasic calcium phosphate through a #40 mesh sieve and transfer to a fluid bed processor.

2. Dissolve Eudragit® RSPO and Eudragit® RLPO in methanol.

3. Granulate the mixture of step 1 with step 2 solution in the fluid bed processor at 50° C., and dry the granules at 50° C.

4. Sift dried granules through a #30 mesh sieve.

5. Sift talc and magnesium stearate through a #60 mesh sieve.

6. Combine the granules and the material of step 5 and blend.

7. Compress the blend of step 6 into tablets.

8. Coat the tablets with an aqueous dispersion of Opadry®, and dry.

### Examples 14B and 14C

1. Sift together drug premix, Kollidon® SR, microcrystalline cellulose, and dibasic calcium phosphate through a #40 mesh sieve.

2. Sift talc and magnesium stearate through a #60 mesh sieve.

3. Combine the materials of steps 1 and 2, and blend.

4. Compress the blend of step 3 into tablets.

5. Coat the tablets with an aqueous dispersion of Opadry®, and dry.

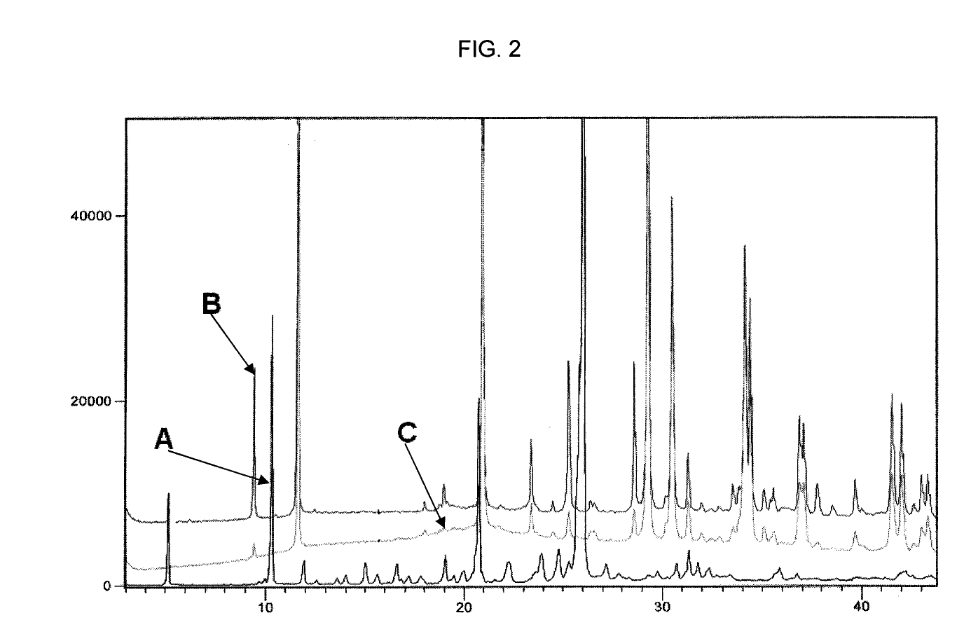

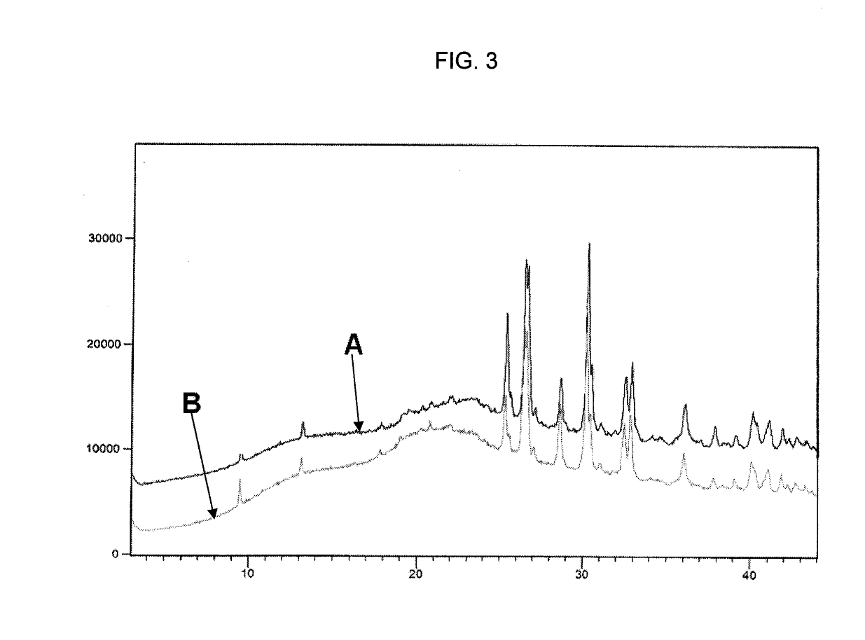

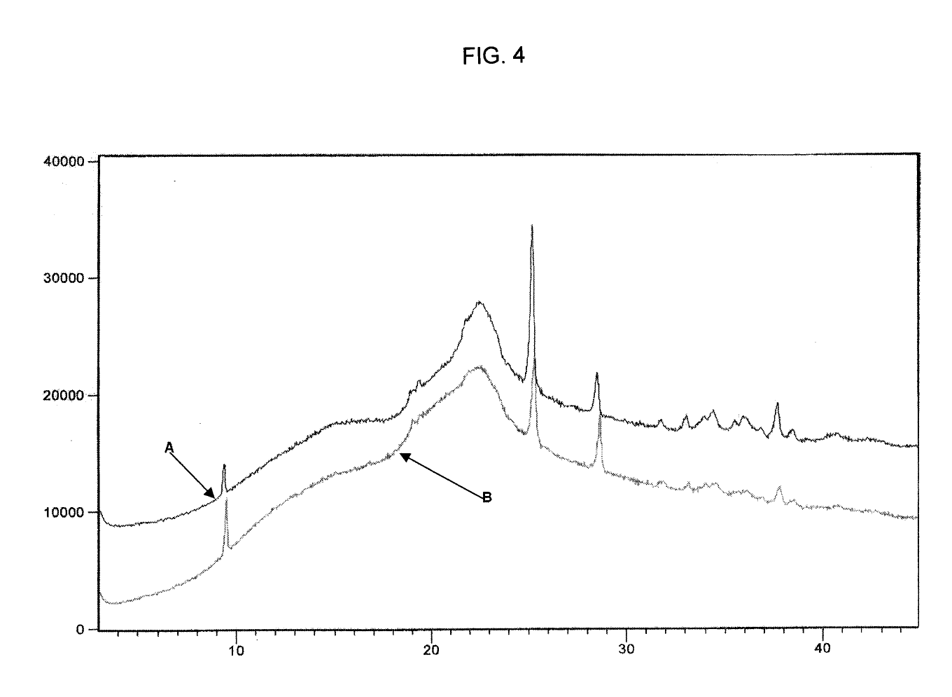

Tablets are stored in an open container at 40° C. and 75% RH and analyzed by PXRD. FIG. 2 shows patterns for tablets of Example 14A (“C”) after storage for three months, placebo tablets prepared similarly, but omitting the desvenlafaxine succinate-PVP premix (“B”), and crystalline desvenlafaxine (“A”). FIG. 3 shows patterns for tablets of Example 14B (“B”) after storage for two months, and placebo tablets prepared similarly, but omitting the desvenlafaxine succinate-PVP premix (“A”). FIG. 4 shows patterns for tablets of Example 14C (“B”) after storage for one month, and placebo tablets prepared similarly, but omitting the desvenlafaxine succinate-PVP premix (“A”). No polymorphic conversion is observed for tablets of Examples 14A-14C.

### Example 15

### Desvenlafaxine Tablets

| Ingredient | 15A | 15B | 15C |
|---|---|---|---|
| Desvenlafaxine succinate-povidone premix | 289.7 | 289.7 | 289.7 |
| Kollidon ® SR | 300 | 383.8 | 358.8 |
| Microcrystalline cellulose (Avicel ™ PH112) | — | 70 | 62.7 |
| Microcrystalline cellulose (Avicel ™ PH102) | 14.1 | — | — |
| Talc | 4 | 4 | 4 |
| Colloidal silicon dioxide (Aerosil ®) | — | — | 7.3 |
| Magnesium stearate | 2.5 | 2.5 | 2.5 |
| Opadry ® II 85F23452 | 30.5 | 37.5 | 21.7 |
| Water* | q.s. | q.s. | q.s. |

*Evaporates during processing.

Manufacturing Procedure:

1. Sift together drug premix, Kollidon® SR, and microcrystalline cellulose through a #40 mesh sieve.

2. Sift talc, magnesium stearate, and Aerosil® (if required) through a #60 mesh sieve.

3. Combine the materials of steps 1 and 2, and blend.

4. Compress the blend of step 3 into tablets.

5. Coat the tablets with an aqueous dispersion of Opadry®, and dry.

Tablets of Examples 15A, 15B, and 15C are processed under different ambient conditions. Tablets of Examples 15A and 15C are processed at 30° C. and RH higher than 40% (i.e., from 45 to 50%), while tablets of example 15B are processed at 25° C. and RH lower than 40% (i.e., from 34 to 37%).

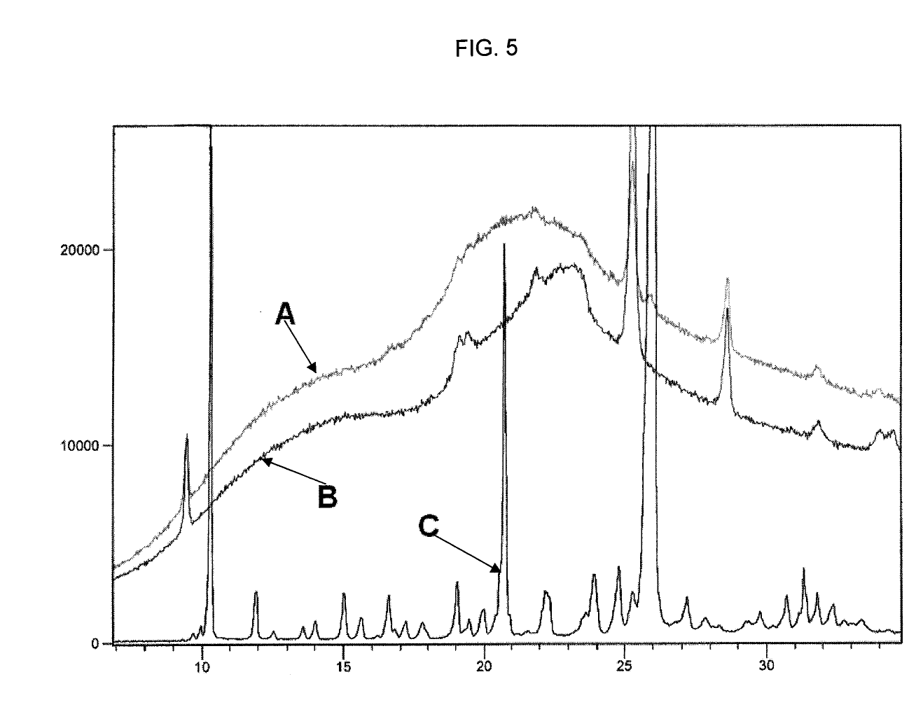

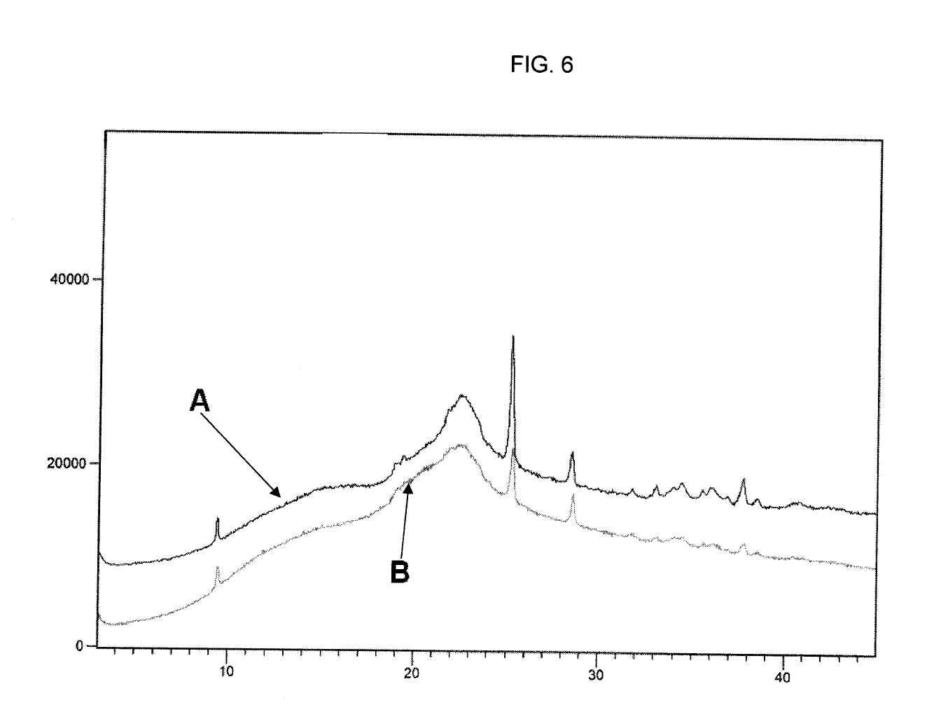

Tablets are analyzed using PXRD. FIG. 5 shows patterns for tablets of Example 15A (“A”), placebo tablets prepared similarly, but omitting the desvenlafaxine succinate-PVP premix (“B”), and crystalline desvenlafaxine succinate (“C”). FIG. 6 shows patterns for tablets of Example 15B (“BA”), and placebo tablets prepared similarly, but omitting the desvenlafaxine succinate-PVP premix (“A”). Polymorphic conversion of the drug is observed in tablets of Example 15A; while tablets of example 15B show polymorphic stability. Thus, it is inferred that processing at lower humidity conditions imparts greater polymorphic stability to the tablet formulations.

### Example 16

### Desvenlafaxine Tablets

| Ingredient | 16A | 16B |
|---|---|---|
| Desvenlafaxine succinate-povidone premix | 289.7 | 289.7 |
| Kollidon ® SR | 358.8 | 358.8 |
| Microcrystalline cellulose (Avicel ™ PH112) | 70 | 70 |
| Talc | 4 | 4 |
| Magnesium stearate | 2.5 | 2.5 |

Subcoating

| | | |
|---|---|---|
| HPMC 5-6 cP | 16.3 | 16.3 |
| Hydroxypropyl cellulose | 5.4 | 5.4 |
| Dichloromethane* | q.s. | q.s. |
| Isopropyl alcohol* | q.s. | q.s. |

Film Coating

| | | |
|---|---|---|
| Opadry ® II 85F23452 | 14 | 14 |
| Water* | q.s. | q.s. |

*Evaporates during processing.

### Manufacturing Procedures

### Example 16A

1. Sift together drug premix, Kollidon® SR, and microcrystalline cellulose through a #40 mesh sieve and transfer to a fluid bed processor.

2. Dry the material of step 1 at 55° C.

3. Sift talc and magnesium stearate through a #60 mesh sieve.

4. Blend the materials of steps 2 and 3.

5. Compress the blend of step 4 into tablets.

6. Coat the tablets with a solution of the subcoating ingredients, using a pan coater, and dry.

7. Coat the tablets of step 6 with an aqueous dispersion of Opadry®, and dry.

### Example 16B

1. Sift together drug premix, Kollidon® SR, and microcrystalline cellulose through a #40 mesh sieve.

2. Sift talc and magnesium stearate through a #60 mesh sieve.

3. Blend the mixtures of steps 1 and step 2.

4. Compress the blend of step 3 into tablets.

5. Coat the tablets using a solution of the subcoating ingredients in a pan coater, and dry.

6. Coat the tablets of step 6 with an aqueous dispersion of Opadry®, and dry.

The granules of Examples 16A and 16B are analyzed for their moisture content. Loss on drying (LOD) values of 1.7% and 2.9% are obtained for the granules of Examples 16A and 16B, respectively.

In-vitro dissolution studies are performed using tablets of Examples 16A and 16B under the following conditions, and the results are as shown in Table 5:

Medium: 0.9% NaCl in water.

Volume: 900 mL.

Apparatus: USP type 1.

Speed: 100 rpm.

Temperature: 37±0.5° C.

TABLE 5

Cumulative
% of Drug
Dissolved

| Hours | 16A | 16B |
|---|---|---|
| 0 | 0 | 0 |
| 1 | 25 | 20 |
| 2 | 31 | 28 |
| 4 | 44 | 40 |
| 6 | 56 | 50 |
| 8 | 68 | 59 |
| 10 | 79 | 67 |
| 12 | 88 | 76 |
| 16 | 102 | 91 |
| 20 | 108 | 101 |
| 24 | 109 | 105 |

### Example 17

### Desvenlafaxine Tablets

| Ingredient | 17A | 17B |
|---|---|---|
| Desvenlafaxine succinate-povidone premix | 289.67 | 289.7 |
| Eudragit ® RS PO | 89 | 89 |
| Eudragit ® RL PO | 25.3 | 25.6 |
| Microcrystalline cellulose (Avicel ™ PH102) | 90 | — |
| Microcrystalline cellulose (Avicel ™ PH112) | — | 140 |
| Methanol* | q.s. | q.s. |
| Talc | 4 | 4 |
| Magnesium stearate | 2 | 2 |

Film Coating

| | | |
|---|---|---|
| Opadry ® II 85F23452 | 15 | 15 |
| Water* | q.s. | q.s. |

*Evaporates during processing.

### Manufacturing Procedures

### Example 17A

1. Sift together drug premix, Eudragit® RSPO, Eudragit® RL PO, and microcrystalline cellulose through a #40 mesh sieve, and blend.

2. Sift talc and magnesium stearate through a #60 mesh sieve.

3. Combine the materials of steps 1 and 3 and blend.

4. Compress the blend of step 3 into tablets.

5. Coat the tablets with an aqueous dispersion of Opadry®, and dry.

### Example 17B

1. Sift together drug premix and dibasic calcium phosphate through a #40 mesh sieve and transfer to a fluid bed processor.

2. Dissolve Eudragit® RSPO and Eudragit® RLPO in methanol.

3. Granulate the materials of step 1 with step 2 solution in the fluid bed processor at 50-60° C., and dry the granules in the fluid bed processor at 50° C.

4. Sift dried granules through a #30 mesh sieve.

5. Sift talc and magnesium stearate through a #60 mesh sieve.

6. Blend the granules with materials of step 5.

7. Compress the blend of step 6 into tablets.

8. Coat the tablets with an aqueous dispersion of Opadry®, and dry.

In-vitro dissolution studies are performed using the tablets and the following conditions, and the results are as shown in Table 6.

Medium: 0.9% NaCl in water.

Volume: 900 mL.

Apparatus: USP type 1.

Speed: 100 rpm.

Temperature: 37±0.5° C.

TABLE 6

Cumulative
% of Drug
Dissolved

| Hours | 17A | 17B |
|---|---|---|
| 0 | 0 | 0 |
| 1 | 62 | 25 |
| 2 | 93 | 35 |
| 4 | 96 | 50 |
| 6 | 96 | 61 |
| 8 | 97 | 70 |
| 10 | 96 | 78 |
| 12 | 96 | 84 |
| 16 | 96 | 92 |
| 20 | 98 | 99 |
| 24 | 98 | 100 |

### Example 18

### Swelling Study

A swelling study is performed using PRISTIQ® 100 mg tablets, tablets of Example 16A, and Tablets of Example 13B. The samples are immersed in 900 mL of saline solution (0.9% NaCl in water) using USP type 1 dissolution testing apparatus at 37±0.5° and a speed of 100 rpm. The dimensions length (L) and width (VV) are measured in millimeters before and at intervals during the test. Results are as shown in Table 7, where the final row is a description of the tablet properties at the end of the test. Tablets of Examples 16A and 13B exhibit less swelling, as compared to the PRISTIQ® tablets.

TABLE 7

| | Length × Width | | |
|---|---|---|---|
| Hours | PRISTIQUE | EXAMPLE 16A | EXAMPLE 13B |
| 0 | 9 × 5 | 13.1 × 6 | 13.1 × 5 |
| 1 | 11 × 6 | 13.1 × 7 | 13.1 × 6 |
| 2 | 11 × 7 | 13.1 × 7 | 13.1 × 6 |
| 3 | 11 × 8 | 14 × 7 | 13.1 × 6 |
| 4 | 12 × 9 | 14 × 8 | 14 × 7 |
| 5 | 12 × 10 | 15 × 8 | 14 × 7 |
| 6 | 12 × 10 | 15 × 9 | 14 × 7 |
| 7 | 12 × 10 | 15 × 9 | 14 × 7 |
| 8 | 13 × 10 | 15 × 9 | 14 × 7 |
| 10 | 13 × 10 | 15 × 9 | 14 × 7 |
| 12 | 13 × 10 | 15 × 9 | 14 × 7 |
| 24 | 13 × 13 | 18 × 10 | 14 × 7 |
| | Gel-like sticky | Spongy, rubber- | Soft palpable |
| | mass | like mass | mass |

We claim:

1. A pharmaceutical formulation comprising: a) amorphous desvenlafaxine in the form of a premix or solid dispersion with a polymer; and b) one or more drug release rate controlling polymers.

2. The pharmaceutical formulation of claim 1, wherein the amorphous desvenlafaxine is present in the form of a solid dispersion with a polyvinylpyrrolidone.

3. The pharmaceutical formulation of claim 1, wherein a drug release rate controlling polymer is a pH independent polymer, a pH dependent polymer, or a combination thereof.

4. The pharmaceutical formulation of claim 1, wherein a drug release rate controlling polymer is one or more of a polyvinyl alcohol, polyvinyl ether, polyvinyl ester, polyvinyl halide, polyvinylpyrrolidone, polyvinyl acetate, polyvinyl alcohol, polyglycolide, polysiloxane, polyurethane or a copolymer thereof, polymer or copolymer of acrylic and methacrylic acid, cellulose acetate butyrate, cellulose acetate phthalate, hydroxypropyl methylcellulose phthalate, and poly(methyl methacrylate).

5. The pharmaceutical formulation of claim 1, wherein a drug release rate controlling polymer is a combination of two different ammonioalkyl methacrylate polymers.

6. The pharmaceutical formulation of claim 1, wherein a drug release rate controlling polymer is one or more ammonioalkyl methacrylate polymers, in combination with a mixture of a polyvinyl acetate and a polyvinylpyrrolidone.

7. The pharmaceutical formulation of claim 1, wherein the weight ratio of desvenlafaxine to a drug release rate controlling polymer ranges from about 1:1 to about 1:10.

8. The pharmaceutical formulation of claim 1, wherein the total drug-related impurities are less than 0.5% of the label content of desvenlafaxine.

9. The pharmaceutical formulation of claim 1, wherein the pharmaceutical formulation comprises desvenlafaxine-containing cores having a coating thereupon comprising one or more polymers.

10. The pharmaceutical formulation of claim 1, wherein the pharmaceutical formulation comprises multi-particulates comprising desvenlafaxine-containing cores coated with one or more polymers in amounts to form portions having different release profiles.

11. The pharmaceutical formulation of claim 1, wherein the pharmaceutical formulation comprises desvenlafaxine coated onto non-pareil cores, being further coated with one or more polymers.

12. A pharmaceutical formulation comprising: a) amorphous desvenlafaxine succinate in the form of a premix or solid dispersion with a polyvinylpyrrolidone; and b) one or more drug release rate controlling polymers.

13. The pharmaceutical formulation of claim 12, wherein a drug release rate controlling polymer is a combination of two different ammonioalkyl methacrylate polymers.

14. The pharmaceutical formulation of claim 12, wherein a drug release rate controlling polymer is one or more ammonioalkyl methacrylate polymers, in combination with a mixture of a polyvinyl acetate and a polyvinylpyrrolidone.

15. The pharmaceutical formulation of claim 12, in the form of a tablet.

16. A process for preparing a pharmaceutical formulation, comprising combining: a) a solid dispersion or premix composition of desvenlafaxine and a polymer; b) at least one drug release controlling substance; and c) optionally, one or more additional pharmaceutically acceptable excipients; and forming the mixture into a dosage form.

17. The process of claim 16, wherein a solid dispersion or premix composition comprises desvenlafaxine succinate and a polyvinylpyrrolidone.

18. The process of claim 16, wherein a drug release controlling substance is a combination of two different ammonioalkyl methacrylate polymers.

19. The process of claim 16, wherein a drug release controlling substance is one or more ammonioalkyl methacrylate polymers, in combination with a mixture of a polyvinyl acetate and a polyvinylpyrrolidone.

20. The process of claim 16, wherein a dosage form is a tablet.
